SEQUENCE LISTING (92 sequences)

SEQ ID NO: 1 - DNA, 3186 bp, E. coli
  CDS (1)..(3186): E. coli rne (wild type) encoding RNase E
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaa

SEQ ID NO: 2 - protein, 1061 aa, E. coli
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKSAPATPAAPAQPGLLSRFFGALKALFSGGEETKPSEQPTPKAEAKPERQQDRRKPRQNNRRDRNERRDTRSERTEGSDNREENRRNRRQAQQQTAETRESRQQVEVTEKARTTDEQQAPRRERSRRRNDDKRQAQQEAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 3 - DNA, 3240 bp, Pseudomonas putida
  CDS (1)..(3240): P. putida rne (wild type) encoding RNAse E
atgaaaagaatgctgattaacgcaactcaacccgaagagttgcgtgtagccctggtggacggccaacgcctctacgacctggacatcgagtccggcgcacgtgagcagaaaaaggccaacatctacaaaggcaagatcacccgcatcgaacccagcctcgaagccgccttcgtcgacttcggttccgaacgtcacggcttcctgccgctgaaagaaatctcccgcgaatacttcaagaaagcccccgaaggccgggtgaacatcaaggaagtgctcagcgaaggccaggaagtcatcgtccaggtcgagaaggaagagcgcggcaacaaaggcgccgccctcaccaccttcatcagcctggctggccgctacctggtgctgatgcccaacaacccgcgtgctggcggcatctcccgccgcatcgaaggcgaagagcgcaacgaactgcgcgaagccctgaacggcctgaccgtgccgggcgacatgggcctgatcgtgcgcactgccggccttggccgcagcagcgaagaaatgcagtgggacctcgactacctgctgcagctgtggaccgccatcaaggaagcatccctggaccgcgccgcgccattcctgatctaccaggaaagcaacgtcatcatccgcgccatccgcgactacctgcgccaggacatcggtgaagtgctgatcgacagcatcgacgcccaggaagaggccctgaccttcatccgccaggtgatgccgcagtacgccagcaaggtgaaactgtacgaagacagcgtaccgctgttcaaccgcttccagatcgaaagccagatcgaaaccgccttccagcgcgtggtcgacctgccgtccggtggttcgatcgtgatcgacccgaccgaagccctggtgtctatcgacatcaactcggcgcgcgccaccaaaggcagcgatatcgaagaaaccgccctgcagaccaacctggaagcggcggaggaaatcgcccgccagctgcgcctgcgtgacatcggcggcctgatcgtgatcgacttcatcgacatgaccccggcgaaaaaccagcgcgccgttgaagaacgcgtgcgcgaatgcctggaagcggaccgtgcccgcgtgcaggttggccgcatctcgcgcttcggcctgctggaaatgtcccgtcagcgcctgcgcccatcgctgggcgaaagcagcggcatcgtctgcccacgctgctccggcaccggcatcatccgtgacgtggagtcgctgtcgctggccatcctgcgcctgatcgaagaagaagccctgaaggaccgcactgccgaagtccgtgcacaagtgccaatcccggtggccgcgttcctgctcaacgagaagcgcaactcgatcaccaagatcgaactgcgcacccgtgcgcgcatcatcatcctgccgaacgatcacctggaaactccgcacttcgaagtccagcgcctgcgcgacgacaacccggaagtgctgaacaaccagtccagctacgagatcgccaccaccgaagccgaagaggcaccgcagcagaccgccacccgcaccctggttcgccaggaagcagcggtcaagaccgccccggcccgcaccaacgcgccggtaccggttgctgaagagcctcaggctgttgccccggccgccccggccccgagcgccccggagccaagcctgttcaaaggcctggtgaagtcgctggtcagcctgttcgccggcaaggacgaacctgcagctgctgcacctgtcgtcgccgagaaaccggccgccgagcgcagcccgcgcaacgaggagcgccgcaacggccgtcaacagagccgcaaccgcaacggccgccgcgacgaagagcgcaagccgcgtgaagagcgtgccgagcgcgccccgcgcgaagagcgccagccacgcgaggagcgtgcaccgcgcgaagagcgtgcaccacgcgaagaacgcgccccacgcgaagagcgtgcaccacgccagccacgcgaagaccgccgcagcaaccgcggcgaagagcgcgtgcgcgaactgcgtgagccgctggatgccaccccgccagccgaacgcgaagagcgccagccacgtgaagagcgtgtagcccgtgaagaacgcgccccacgtgaagagcgtgcacctcgcgaagaacgtgctccgcgtgaagagcgcgcacctcgcgaagaacgtgctccgcgtgaagagcgtgctcctcgcgaagaacgtgctccgcgtgaagagcgcgcacctcgcgaagaacgtgcgccacgcgaagaacgcgccccgcgcccaccacgcgaagagcgccagccacgcgtagccgaagaagcggccgagcaggctgccgaactggccgaagagcaactgccgaacgaagagctgctgcaagacgaacaggaaggcaccgatggcgagcgtccgcgccgccgctcccgtggccagcgtcgtcgcagcaaccgtcgtgagcgccagcgcaacgccaatggcgagctgatcgacggtggcgaagaggaagaaggcagcgaagagcagccacaacagcaccaggccaccgagctgggtgccgaactggccgccggcctggcagtgactgccgctgttgccagcagcaacatcagcgccgacgccgaagcccaggccaaccagcaggccgaactggccaccgccgaaatcgctgccgcagcagagaccgacaacagccacgccgctcagccggtcgagaaggctgaaaaggtcgagccggttgaagccgtcgccaaggctgaagacgttgccgtagccccagtggtggagcagcctgtcagcgagccggttgtcgtggccgaggtcactgccgagccggtagtcgaagtcgctccgcaacacctggttgaagaagcgcctgctgccgaaccggtaatcgtcgccgaagcccctgttgaaacgcctgctgtcgaagcgggcgaaatcgaaaaagctccggccgtggttgaaaccgctccggttgccgagcagcctgcaccggttgtcgaagcccagccagaagtagtcgcagagcctgcacctgtggtcgtcgagcctgccccggtagaggcagaagctgccaccgtcatgctggccaacggccgtgcgccgaacgacccgcgtgaagtgcgccgccgcaagcgtgaggccgaggccgccgccaaagccgcgcaggaagctgctgcagcgaccgaggagccagccctggaagccgccgatgagcacaagcctcatcacagttga

SEQ ID NO: 4 - protein, 1079 aa, Pseudomonas putida
MKRMLINATQPEELRVALVDGQRLYDLDIESGAREQKKANIYKGKITRIEPSLEAAFVDFGSERHGFLPLKEISREYFKKAPEGRVNIKEVLSEGQEVIVQVEKEERGNKGAALTTFISLAGRYLVLMPNNPRAGGISRRIEGEERNELREALNGLTVPGDMGLIVRTAGLGRSSEEMQWDLDYLLQLWTAIKEASLDRAAPFLIYQESNVIIRAIRDYLRQDIGEVLIDSIDAQEEALTFIRQVMPQYASKVKLYEDSVPLFNRFQIESQIETAFQRVVDLPSGGSIVIDPTEALVSIDINSARATKGSDIEETALQTNLEAAEEIARQLRLRDIGGLIVIDFIDMTPAKNQRAVEERVRECLEADRARVQVGRISRFGLLEMSRQRLRPSLGESSGIVCPRCSGTGIIRDVESLSLAILRLIEEEALKDRTAEVRAQVPIPVAAFLLNEKRNSITKIELRTRARIIILPNDHLETPHFEVQRLRDDNPEVLNNQSSYEIATTEAEEAPQQTATRTLVRQEAAVKTAPARTNAPVPVAEEPQAVAPAAPAPSAPEPSLFKGLVKSLVSLFAGKDEPAAAAPVVAEKPAAERSPRNEERRNGRQQSRNRNGRRDEERKPREERAERAPREERQPREERAPREERAPREERAPREERAPRQPREDRRSNRGEERVRELREPLDATPPAEREERQPREERVAREERAPREERAPREERAPREERAPREERAPREERAPREERAPREERAPREERAPREERAPRPPREERQPRVAEEAAEQAAELAEEQLPNEELLQDEQEGTDGERPRRRSRGQRRRSNRRERQRNANGELIDGGEEEEGSEEQPQQHQATELGAELAAGLAVTAAVASSNISADAEAQANQQAELATAEIAAAAETDNSHAAQPVEKAEKVEPVEAVAKAEDVAVAPVVEQPVSEPVVVAEVTAEPVVEVAPQHLVEEAPAAEPVIVAEAPVETPAVEAGEIEKAPAVVETAPVAEQPAPVVEAQPEVVAEPAPVVVEPAPVEAEAATVMLANGRAPNDPREVRRRKREAEAAAKAAQEAAAATEEPALEAADEHKPHHS

SEQ ID NO: 5 - DNA, 1563 bp, Bacillus subtilis
  CDS (1)..(1563): B. subtilis rny (wild type) encoding RNase Y
atgaccccaattatgatggttctcatctccattttgctgattctactcggtttagttgttggctactttgttcgtaaaaccattgccgaagcgaaaattgcgggcgcacgcggtgcagccgagcaaattcttgaagatgcgaagcgtgatgctgaagcactgaaaaaagaagctctgcttgaagcaaaggatgaaatccacaaacttcgaatagatgctgaacaggaagttcgtgaaagacgaaatgagcttcaaaaacaagaaaaccgtttactccaaaaggaggaaaaccttgatcgcaaacatgagggaattgataaacgggaagcgatgttggagaagaaagatcattctctgaatgaacgacaacaacatattgaagagatggaaagcaaagtggatgagatgattcgtatgcagcagtctgagttggaacgaatttcgagtctgactcgtgacgaagcgaaacaaatcattcttgagcgggttgaaaacgagctttcacatgacatcgccatcatgacaaaagaaactgaaaaccgtgcgaaagaagaggcggataagaaagcgaaaaacattctttcactcgccttacagcgctgcgcagcggaccacgttgccgaaacaacggtatcagttgtcaatcttccaaatgatgagatgaaaggacgtatcatcggacgggaagggcgtaacattcgtacgcttgaaacgctgacaggaattgacctgattattgatgatacgcctgaagctgtcattctttccggatttgatccgatcagacgtgagacagccagaattgctcttgataaactcgttcaggatggccgtattcatccggcacggattgaagaaatggttgaaaaatctcgccgcgaggtcgatgactatattcgtgagatgggtgagcaaacgacatttgaggttggcgttcatggcctccacccagatctcatcaagatcctcggccgcttaaagttccgtacaagctacggtcaaaatgtgcttaagcattccatggaagtcgcattcttggccggtctaatggcatcggagcttggtgaagacgcaaagcttgctaaacgtgcgggtcttcttcacgacatcgggaaagcaattgaccatgaagtagaaggaagccacgttgagatcggggtagagcttgcgaccaaatataaagagcacccagtcgtgattaacagtattgcatcacaccacggggacgaggagccgacttccattattgctgtactggtagctgcagcagatgcgctttccgctgcaagacctggcgcaagaagtgagacgctcgagaattatattcgaagacttgaaaaacttgaagaaatttctgagtcctacgaaggtgttgaaaaatcatttgccattcaggctggacgcgaagtgcgaattatggtgaagccggattcaattaatgatcttgaggctcatcgactggcgcgagatatccgcaagcgaattgaggacgagctcgattatccaggtcatattaaggtgacagtaatcagagagactcgagccgtagagtatgcaaaataa

SEQ ID NO: 6 - protein, 520 aa, Bacillus subtilis
MTPIMMVLISILLILLGLVVGYFVRKTIAEAKIAGARGAAEQILEDAKRDAEALKKEALLEAKDEIHKLRIDAEQEVRERRNELQKQENRLLQKEENLDRKHEGIDKREAMLEKKDHSLNERQQHIEEMESKVDEMIRMQQSELERISSLTRDEAKQIILERVENELSHDIAIMTKETENRAKEEADKKAKNILSLALQRCAADHVAETTVSVVNLPNDEMKGRIIGREGRNIRTLETLTGIDLIIDDTPEAVILSGFDPIRRETARIALDKLVQDGRIHPARIEEMVEKSRREVDDYIREMGEQTTFEVGVHGLHPDLIKILGRLKFRTSYGQNVLKHSMEVAFLAGLMASELGEDAKLAKRAGLLHDIGKAIDHEVEGSHVEIGVELATKYKEHPVVINSIASHHGDEEPTSIIAVLVAAADALSAARPGARSETLENYIRRLEKLEEISESYEGVEKSFAIQAGREVRIMVKPDSINDLEAHRLARDIRKRIEDELDYPGHIKVTVIRETRAVEYAK

SEQ ID NO: 7 - DNA, 1560 bp, Bacillus licheniformis
  CDS (1)..(1560): Bacillus licheniformis rny encoding RNase Y
atgagtcctttagcaattctcatctccattttgctgagcctattctgtttagttgttggctactatgttcgtaaaatcattgccgaagcaaaaatttcaggtgcgcgaaatgcagccgaacaaattcttggagacgcaaagcgggatgctgaagcgttgaaaaaagaagcccttcttgaagcaaaggacgagattcatacgcttcggatagaagctgaacaagaagttcgtgaaagacgaaatgagcttcaaaaacaagaaaaccgtttacttcaaaaggaagagaaccttgaccgaaaagatgaatcattagataaacgggaagcgatgttggagaagaaagatcattctctgaatgaacgacaacaacatattgaagagatggaaagcaaagtggatgaaatgattcgtatgcagcagtcagagttggagcgtatttcaagtctgacgcgagatgaagcgaagcaaatcattctggaacgggttgaaaacgagctttcccatgacatcgcgatcatgatgaaagaaagtgaaaatcgagcgaaagaagaggctgataaaaaggcgaaaaatattctttcattggctttacagcgctgtgcggcagatcatgtggccgagacaacggtttcagttgtcaaccttccaaatgatgagatgaaaggccgcattatcggacgtgaaggacgtaatatccgtacattggaaacattgacggggatcgatctgatcatcgacgatacgccggaagccgttatcctttcaggatttgatccgatcaggcgtgaaacagccaggatcgctcttgataaactcgttcaggatggccgcattcatcctgccagaatcgaagaaatggttgagaaatcccgccgtgaagtggatgattacatccgcgaaatgggtgaacagacgacgtttgaagttggagttcacggtcttcatcccgatttaatcaaaatcctcggccgcttgaagttcagaacgagctacggacagaatgtactgaaacattcaatggaagtggcgtttctgacaggcttgatggcttcagagctcggagaagacgtaacgctcgcaaaaagggcaggacttcttcatgatatcgggaaagcgattgaccatgaggtggaaggaagccacgttgaaatcggtgtggaactggccaccaagtataaagagcatccagttgtcatcaacagtatcgcatctcaccacggagatcaggagccgacttccatcatcgccgtgctcgtggccgcagccgatgcgctgtctgccgcaagaccaggcgcaagaagcgaaacgctcgaaaactatattcgcaggcttgaaaaactggaagaaatctccgaatcatacgaaggtgttgaaaaatcattcgcgatacaagcgggacgagaggtgcggatcatggtcaaacctgactcaatcaatgatcttgaagcccatcgtttggcgcgtgatattcgaaaacgaattgaggacgagcttgactaccctggacacatcaaggtaaccgtaatcagggaaacgagagccgttgaatacgcaaaataa

SEQ ID NO: 8 - protein, 519 aa, Bacillus licheniformis
MSPLAILISILLSLFCLVVGYYVRKIIAEAKISGARNAAEQILGDAKRDAEALKKEALLEAKDEIHTLRIEAEQEVRERRNELQKQENRLLQKEENLDRKDESLDKREAMLEKKDHSLNERQQHIEEMESKVDEMIRMQQSELERISSLTRDEAKQIILERVENELSHDIAIMMKESENRAKEEADKKAKNILSLALQRCAADHVAETTVSVVNLPNDEMKGRIIGREGRNIRTLETLTGIDLIIDDTPEAVILSGFDPIRRETARIALDKLVQDGRIHPARIEEMVEKSRREVDDYIREMGEQTTFEVGVHGLHPDLIKILGRLKFRTSYGQNVLKHSMEVAFLTGLMASELGEDVTLAKRAGLLHDIGKAIDHEVEGSHVEIGVELATKYKEHPVVINSIASHHGDQEPTSIIAVLVAAADALSAARPGARSETLENYIRRLEKLEEISESYEGVEKSFAIQAGREVRIMVKPDSINDLEAHRLARDIRKRIEDELDYPGHIKVTVIRETRAVEYAK

SEQ ID NO: 9 - DNA, 2514 bp, Saccharomyces cereviciae
  CDS (1)..(2514): CCR4
atgaacgacccttctttactaggctaccctaatgtgggcccgcagcagcaacagcaacagcagcaacagcagcatgccgggctactaggaaagggtacaccaaacgctctacagcagcagctacacatgaaccagctcaccgggatacctccaccgggactcatgaacaacagcgatgtacacacttccagcaacaacaactcgcgccagttgctcgaccaattggctaatgggaacgcgaacatgctcaatatgaacatggataacaacaataacaacaataacaacaacaacaacaacaacaacaacggcggtggaagcggggtgatgatgaatgcctccacagccgcagtaaattctatagggatggttcctaccgttggtacgcctgtgaacatcaatgtgaacgccagtaaccctttattacacccgcacttggatgacccttctttactaaacaatccgatctggaagcttcaattgcatctggctgcagtgtccgcacaatctctggggcaacccaacatttatgctagacaaaacgctatgaagaagtatttggctacacaacaggctcagcaagctcagcaacaagcgcagcaacaggcacagcagcaggtcccgggcccatttggccccggacctcaggctgcaccaccagctttgcagcccaccgatttccagcaatctcacattgcagaagcctccaaatcactggtagactgcacaaagcaagccttgatggaaatggccgacactctcaccgacagcaagacagcaaagaaacaacaacctacgggagatagcactccctcaggcacggcaactaacagtgcagtttctacaccattgactcccaagatagagctgtttgctaatggcaaggacgaagccaaccaggcgctcttacaacacaagaaactgtctcagtacagcatcgacgaagatgacgacattgaaaacagaatggtcatgcccaaggactcgaaatacgacgaccaattatggcacgcgctagatttgtccaacttgcaaatcttcaatatcagcgccaacatcttcaagtacgattttctaacgagactatatttgaatggcaatagcctcacggaactgccagcggagatcaagaacctaagcaacctacgcgttttggacctgtcgcataataggttaacatctctaccagcggaactaggctcatgtttccaattgaaatacttctacttttttgataacatggtcaccacattaccatgggagtttgggaacctgtgtaaccttcagtttcttggtgtagaaggaaaccccttagaaaaacagtttttaaagatccttacagaaaaatctgtcacgggattgattttctacctaagggacaacagaccagagattcccttaccgcatgaacgtaggttcatcgaaatcaataccgatggggaaccacagagggagtacgattctttgcagcaatccactgagcatctggccaccgatttagccaagaggacgttcaccgtcttatcctacaacaccttatgtcaacactatgccaccccaaaaatgtaccgttacacaccgtcgtgggcgttaagttgggattacaggcgcaataaattaaaggagcagattctctcgtacgacagtgatctgttgtgtttacaagaagtggagtctaagacttttgaagagtattgggtgcccctattggacaagcacggttatacaggcatcttccatgcaaaggcaagagccaagaccatgcattccaaggactccaagaaagtggacgggtgttgcatttttttcaaaagggaccaattcaagttgatcaccaaagacgccatggatttcagcggtgcttggatgaaacacaagaagttccaaagaactgaagattatttaaaccgtgcaatgaacaaagacaacgttgcactgttcttaaagctacaacacattcctagtggcgacaccatatgggcggtcaccacgcatttgcactgggatccaaaatttaatgatgtcaagacattccaagtaggtgtcctgttagatcatctggaaacgctgctaaaggaggagacatcgcacaattttagacaggacattaagaaatttcctgtgctcatttgtggtgacttcaattcatacatcaactccgccgtatacgaattgataaatacaggccgtgtccaaatacatcaagagggaaatggtagagatttcggttacatgtcggagaaaaatttctcacataacttggctctcaaatcaagttataattgtatcggagaactaccattcaccaatttcacaccgtcattcacagatgttatcgactatatatggttttctacacatgctctaagggtgcgtgggctattgggtgaagtggaccctgaatacgtgagtaagtttatcgggttccccaacgacaaattccccagtgaccatataccattgttagcaagatttgaatttatgaagacaaacacaggcagtaagaaagtataa

SEQ ID NO: 10 - protein, 837 aa, Saccharomyces cereviciae
MNDPSLLGYPNVGPQQQQQQQQQQHAGLLGKGTPNALQQQLHMNQLTGIPPPGLMNNSDVHTSSNNNSRQLLDQLANGNANMLNMNMDNNNNNNNNNNNNNNNGGGSGVMMNASTAAVNSIGMVPTVGTPVNINVNASNPLLHPHLDDPSLLNNPIWKLQLHLAAVSAQSLGQPNIYARQNAMKKYLATQQAQQAQQQAQQQAQQQVPGPFGPGPQAAPPALQPTDFQQSHIAEASKSLVDCTKQALMEMADTLTDSKTAKKQQPTGDSTPSGTATNSAVSTPLTPKIELFANGKDEANQALLQHKKLSQYSIDEDDDIENRMVMPKDSKYDDQLWHALDLSNLQIFNISANIFKYDFLTRLYLNGNSLTELPAEIKNLSNLRVLDLSHNRLTSLPAELGSCFQLKYFYFFDNMVTTLPWEFGNLCNLQFLGVEGNPLEKQFLKILTEKSVTGLIFYLRDNRPEIPLPHERRFIEINTDGEPQREYDSLQQSTEHLATDLAKRTFTVLSYNTLCQHYATPKMYRYTPSWALSWDYRRNKLKEQILSYDSDLLCLQEVESKTFEEYWVPLLDKHGYTGIFHAKARAKTMHSKDSKKVDGCCIFFKRDQFKLITKDAMDFSGAWMKHKKFQRTEDYLNRAMNKDNVALFLKLQHIPSGDTIWAVTTHLHWDPKFNDVKTFQVGVLLDHLETLLKEETSHNFRQDIKKFPVLICGDFNSYINSAVYELINTGRVQIHQEGNGRDFGYMSEKNFSHNLALKSSYNCIGELPFTNFTPSFTDVIDYIWFSTHALRVRGLLGEVDPEYVSKFIGFPNDKFPSDHIPLLARFEFMKTNTGSKKV

SEQ ID NO: 11 - DNA, 3348 bp, Saccharomyces cereviciae
  CDS (1)..(3348): Pan2
atgaataattggcaacatttcttcaacaatccagttgatctttcggaacatttgaagaagccatactttcgcttcgataatagggataaggaaattacagcgattagcttcgatgagaaggcaaacttaatttggagtggagacagctatggttgcatttcgtcatatgatccaacttttcaactttatacaagatataggggccacataggtggaaattccgtgaaggatattctcagtcatcgggatggtattttatctattagtgaagattccttacactttgctaatagaagaggtgttactaaattgaacctcactagcattgatattgctgcatttagcgaattgaacactatgtgctattctcctcattcactgaaaaacaatatctactgtggtggtgacaacacaaattggggaattgcgtccattgacttgaacagaggttgcttagattccctcttgaattactcatctaaagtgaagttaatgtgctctaataataaagttttgtctatcggaagacaaacagggactgtggatttgctagatccaacatcgaatcgtactatcaaatcatttaatgcacactctgcatccatatccgctatggatttacgggataacaccttggttacagtagggaagtccaaaagattttataacttatacgctgacccatttgtgaatgtttacgacttgagaacaatgcgtcaactccctcctgtttccttttctaaaggaacaactatgggatctggaggcgcagattttgttcaattacatcctttgcttcctactgttatgatcgtcgcctcaagttctggttcgtttgatttcatcgacctttccaatccaactttaagaacacaatatgttcatccttgccagtcgattaaaaagttatgtttgtcccccaatggtgacgtattgggtatactagaagctgataatcacctagatacatggagaagatcatcaaacaacatgggaatgtttaccaatacccctgaaatgctagcatatcctgattattttaatgacattacctctgacggcccaatatctgtcgacgatgaaacatatccattgagttctgtggggatgccgtactatcttgataaacttttgtctgcatggccccctgtagtgtttaaaagtgaaggtaccataccgcaattaacaggtaagtcacccttaccatcgagcggcaaattaaaaagtaaccttgctgtgatctcgagccaaaatgagaagttgagcacacaagaatttcctttgttaagatatgatcgcaccaaatacggtatgagaaatgctataccagattacgtttgtctaagagatataaggaaacagataacaagcggtttagaaaccagcgatatacagacatatacctcaatcaacaagtacgaagtaccccctgcatacagtagacttccactgacatcaggtagatttggtactgataattttgattttacgccctttaataacactgagtattcaggattggatccagatgttgataatcactacacaaatgctatcatacaattgtatcgctttattccagaaatgtttaatttcgttgttgggtgtttgaaagacgagaattttgaaacaacgttgctaactgatctaggctacctctttgacatgatggaaagatcacatggaaaaatatgtagttcttccaattttcaggcgtcattgaaatccttaactgataaaagacaattagaaaacggtgaaccacaagaacatttagaagagtatttagaatcgctgtgcataagggaaagtatcgaggattttaattcttctgaaagtattaaacgcaatatgcctcaaaaatttaacagattcctgctctcgcaacttattaaagaggaagcgcagacagtcaaccataatatcaccctaaatcaatgctttggtttggaaacggaaatacgaacagagtgtagctgtgatcactacgacactaccgtcaaacttctaccctccttatcaatatcaggaatcaacaaaaccgtaatcaaacaattgaacaagaaaagcaatggacagaatattttgccttatattgaatatgccatgaaaaatgtaacccaaaagaacagtatttgcccaacctgcggcaaaaccgaaactatcacccaggagtgtactgtcaagaatttaccttcagtgttgtcattagaattatcactattagataccgaattttccaatataaggtcgtcgaaaaactggttaactagtgaattttatggaagcatcattaaaaacaaggcagttctaagatcgacggcgtccgaattgaagggcacaagccacatatttaaatacgaattgaatggttacgtggctaaaatcactgataacaataacgagacgcgtctagtaacatatgtcaaaaaatataatccaaaagagaattgcttcaagtggctcatgtttaatgattatttggttgttgagataacagaggaagaggcgcttaaaatgacatacccttggaaaacaccagaaattatcatatattgtgatgcggaagaattacgaaaacctttcttttctgttgatacgtattccatcaactatgacatacttttccgtgattatttcgcaaacggaataagagatactgcaagacgtgaatataagttattaacacatgatgaggcacctaaatctggaaccttggttgccattgatgccgaatttgtctcattacaaagtgaactatgtgaaatcgatcatcaaggaatcagaagtattattcgacctaaaagaactgctttggccagaatatccattattagaggcgaagaaggagaactgtatggagtaccatttgtcgatgattatgtggtaaacacgaaccacatagaagactatttgacaagatatagtgggattcttcctggtgacttggaccctgaaaagagtaccaaaaggcttgtgagaagaaacgttgtatatcgaaaagtctggcttttaatgcagctcggatgcgtatttgttggtcatggtttgaataatgacttcaaacacattaatattaatgtcccaagaaaccaaattcgcgacactgccatatattttctacaaggaaagagatatctttcattgcgttatctggcatatgtgttgttaggaatgaatatccaagagggaaatcacgattcaattgaagatgcccatactgccttgattctttacaaaaaatatctccacctgaaagaaaaagctatctttgagaaagtactgaacagcgtgtacgaagaaggaagagcccataatttcaaagttccagaaacttcaaagggataa

SEQ ID NO: 12 - protein, 1115 aa, Saccharomyces cereviciae
MNNWQHFFNNPVDLSEHLKKPYFRFDNRDKEITAISFDEKANLIWSGDSYGCISSYDPTFQLYTRYRGHIGGNSVKDILSHRDGILSISEDSLHFANRRGVTKLNLTSIDIAAFSELNTMCYSPHSLKNNIYCGGDNTNWGIASIDLNRGCLDSLLNYSSKVKLMCSNNKVLSIGRQTGTVDLLDPTSNRTIKSFNAHSASISAMDLRDNTLVTVGKSKRFYNLYADPFVNVYDLRTMRQLPPVSFSKGTTMGSGGADFVQLHPLLPTVMIVASSSGSFDFIDLSNPTLRTQYVHPCQSIKKLCLSPNGDVLGILEADNHLDTWRRSSNNMGMFTNTPEMLAYPDYFNDITSDGPISVDDETYPLSSVGMPYYLDKLLSAWPPVVFKSEGTIPQLTGKSPLPSSGKLKSNLAVISSQNEKLSTQEFPLLRYDRTKYGMRNAIPDYVCLRDIRKQITSGLETSDIQTYTSINKYEVPPAYSRLPLTSGRFGTDNFDFTPFNNTEYSGLDPDVDNHYTNAIIQLYRFIPEMFNFVVGCLKDENFETTLLTDLGYLFDMMERSHGKICSSSNFQASLKSLTDKRQLENGEPQEHLEEYLESLCIRESIEDFNSSESIKRNMPQKFNRFLLSQLIKEEAQTVNHNITLNQCFGLETEIRTECSCDHYDTTVKLLPSLSISGINKTVIKQLNKKSNGQNILPYIEYAMKNVTQKNSICPTCGKTETITQECTVKNLPSVLSLELSLLDTEFSNIRSSKNWLTSEFYGSIIKNKAVLRSTASELKGTSHIFKYELNGYVAKITDNNNETRLVTYVKKYNPKENCFKWLMFNDYLVVEITEEEALKMTYPWKTPEIIIYCDAEELRKPFFSVDTYSINYDILFRDYFANGIRDTARREYKLLTHDEAPKSGTLVAIDAEFVSLQSELCEIDHQGIRSIIRPKRTALARISIIRGEEGELYGVPFVDDYVVNTNHIEDYLTRYSGILPGDLDPEKSTKRLVRRNVVYRKVWLLMQLGCVFVGHGLNNDFKHININVPRNQIRDTAIYFLQGKRYLSLRYLAYVLLGMNIQEGNHDSIEDAHTALILYKKYLHLKEKAIFEKVLNSVYEEGRAHNFKVPETSKG

SEQ ID NO: 13 - DNA, 2040 bp, Saccharomyces cereviciae
  CDS (1)..(2040): Pan3
atggacaaaatcaatcctgattgggcgaaggatattccgtgcagaaatatcactatttatggctactgcaaaaaggagaaagaaggttgccctttcaaacacagcgataacactaccgctactaccataaatgacgttcctcctccaatagatgtgggtgaggctacaactccgaccatgacatcagttcctaagttcaatgctaaagtatccgcaagtttcactccgatgacagtcggtagtgactccttaaccactgtgacgaataccacctccgctgctacaaatgctactggcaatatcgccatggcagctacctctgctactgcttctacagttaatccgatgattaatccgatagttaatagctcgttagtgaataacaataacaataatagtaatataagcatatcaataccaactaccgcttcgagttccaattacgaccccttcaatgcccccattttcactccgtcttcaacctcctcaattcacactaatgcaaatgcacattcttttccatttccctccattgcaaattctggtggcataaatataaacgccactgatgataatagtaacaatatgagtatggctaataatgtgccacctcctatgcaaccgccacccatagagagtagtaatcttaagtacccacgtatttatccgcctcctcacagtcttctacagtatcacctatatgcacctgaacagccatcatcattgaaatcattattaaagcctaatgaaaggtctgcagatcagcttttcattccaaacaatattagagaagatttaaccaagaaaaacttatcgattttgcaggttttcccctcttcaggtaaagttataccaagtattgtacaagattattttaatttggttccattgaacttcaataataacgattttttaaataaaactacgctcttcaaagttttttccaattatgacggtaaagcctacgttttgaagaggcttcctaacatcgataagtcaatgaatccaaacaaaatatccaaaatatatcagatatggtcaaaaattaattgtacaaatttgataaagtttagggacatttttcaaactactaaatttggtgatctgtctatttgtttggtctttgactactacccaaactcgctatctttgtatgattaccactttgttaatttccctaagtttccaataacgaataattatttatggatatatttagttcaactcaccaatgtaataaactctatccattcacaaaacttgagtattggcaatacattaaactggagaaaagttttgattactggggacccagggagaatcaagttatcacactgcaattttatggaccttttgttcaatgatgatactgataccgtagtatcttccggcggaagtaccatagagggacaacaacagctagactacaaatatttaggagagctattatttaacctatccattaatattgaaaactctaataacaacactgcccctaaagaatatcgattggaggaaataacccctcaatcaattgatgacatgagacagatcgatgataagttcaaggatgtactcaagtatctgatatcagacaacggcgattccaaaaagagcattcatgatcttactagtcacttttatgataagatgttcatggtcctggaatcgtcacaaacctatacagaatacatggagtctgtcttatcaagagaactagaaaatggcagattatttaggctggtcaacaagctaaattgcatttttggtagaatcgaatcaagaatagacataaattggtccgaatctgggactaaattccccattatactattttatgactacgtattccatcaagtggattcgaatgggaaaccaataatggatttaactcatgtcctaagatgtttgaacaaattagacgctggtattcaagaaaagttaatgttggtaacgcccgatgagttaaactgtattattatatcctataaggagttgaaggacttgatagaatccacctttcgatccatcacccaataa

SEQ ID NO: 14 - protein, 679 aa, Saccharomyces cereviciae
MDKINPDWAKDIPCRNITIYGYCKKEKEGCPFKHSDNTTATTINDVPPPIDVGEATTPTMTSVPKFNAKVSASFTPMTVGSDSLTTVTNTTSAATNATGNIAMAATSATASTVNPMINPIVNSSLVNNNNNNSNISISIPTTASSSNYDPFNAPIFTPSSTSSIHTNANAHSFPFPSIANSGGININATDDNSNNMSMANNVPPPMQPPPIESSNLKYPRIYPPPHSLLQYHLYAPEQPSSLKSLLKPNERSADQLFIPNNIREDLTKKNLSILQVFPSSGKVIPSIVQDYFNLVPLNFNNNDFLNKTTLFKVFSNYDGKAYVLKRLPNIDKSMNPNKISKIYQIWSKINCTNLIKFRDIFQTTKFGDLSICLVFDYYPNSLSLYDYHFVNFPKFPITNNYLWIYLVQLTNVINSIHSQNLSIGNTLNWRKVLITGDPGRIKLSHCNFMDLLFNDDTDTVVSSGGSTIEGQQQLDYKYLGELLFNLSINIENSNNNTAPKEYRLEEITPQSIDDMRQIDDKFKDVLKYLISDNGDSKKSIHDLTSHFYDKMFMVLESSQTYTEYMESVLSRELENGRLFRLVNKLNCIFGRIESRIDINWSESGTKFPIILFYDYVFHQVDSNGKPIMDLTHVLRCLNKLDAGIQEKLMLVTPDELNCIIISYKELKDLIESTFRSITQ

SEQ ID NO: 15 - DNA, 666 bp, Saccharomyces cereviciae
  CDS (1)..(666): Rpb4
atgaatgtttctacatcaacctttcaaacaagacggagaagattgaagaaagtggaggaagaagaaaatgcagctactctacaactgggccaggaattccagctgaaacagataaatcatcagggtgaagaggaggaattgattgccttgaacctaagtgaagccaggttagtaatcaaagaagctcttgtagaacgtaggagagcatttaaaagatcgcaaaaaaaacacaagaagaagcatttgaagcacgaaaacgccaatgatgaaactacggcagtagaggatgaagatgatgatctggatgaagatgacgtcaacgctgatgatgatgattttatgcattctgaaactagggagaaggagttggagtctatcgacgttctgttagaacagacaacgggaggaaataataaagatttgaaaaataccatgcagtatttaacaaatttctcccgatttagagaccaagaaaccgtcggggcagttatacagcttctgaaaagcactgggttacatccttttgaagtggcgcaactaggttctttggcctgtgacacagctgatgaagcaaagactttaattccaagcttaaacaataaaatatcagacgatgagttggaaaggatactaaaggaattgtcaaacctagaaacactctattaa

SEQ ID NO: 16 - protein, 221 aa, Saccharomyces cereviciae
MNVSTSTFQTRRRRLKKVEEEENAATLQLGQEFQLKQINHQGEEEELIALNLSEARLVIKEALVERRRAFKRSQKKHKKKHLKHENANDETTAVEDEDDDLDEDDVNADDDDFMHSETREKELESIDVLLEQTTGGNNKDLKNTMQYLTNFSRFRDQETVGAVIQLLKSTGLHPFEVAQLGSLACDTADEAKTLIPSLNNKISDDELERILKELSNLETLY

SEQ ID NO: 17 - DNA, 516 bp, Saccharomyces cereviciae
  CDS (1)..(516): Rpb7
atgttttttattaaagacctttcgcttaatattacccttcatccgtcctttttcggtcctcgaatgaagcaatatctaaagacaaaactattggaagaggttgaaggttcgtgtacgggtaaattcggatatattctttgtgtcctagactatgataatatagatattcaacgtgggagaatattgcccacagatgggtccgccgagttcaacgtgaaatatagagctgtagttttcaaaccatttaaaggggaagtagtggacggcacagtcgtttcatgttctcagcacgggttcgaagtgcaagtaggtccaatgaaagtatttgtgacaaagcatctgatgcctcaagatttaacctttaatgcgggttcaaacccaccatcataccaaagttccgaggatgtcatcaccataaaaagtagaattagagttaaaattgaaggttgtatcagtcaagtgagttctattcacgcaatcggtagtatcaaagaagattatttgggtgctatttaa

SEQ ID NO: 18 - protein, 171 aa, Saccharomyces cereviciae
MFFIKDLSLNITLHPSFFGPRMKQYLKTKLLEEVEGSCTGKFGYILCVLDYDNIDIQRGRILPTDGSAEFNVKYRAVVFKPFKGEVVDGTVVSCSQHGFEVQVGPMKVFVTKHLMPQDLTFNAGSNPPSYQSSEDVITIKSRIRVKIEGCISQVSSIHAIGSIKEDYLGAI

SEQ ID NO: 19 - protein, 743 aa, Pichia pastoris
  peptide (1)..(743): CCR4
MNTASQYKAQGNGQGIMRHAPQPQQTQIPSQYLLQQLQGSHATNQPQTDSSNPSLLVQALFQGPQSHQGQRIGNQQMQPHVVLGSQTLQSSSVVPPGQNQPTSQSPHYDTIQVEDPTTFHWQHQVQLVQMSRKCNQPHFYARHAAVSSRKLLGKNGNNNMPGVDGSQGNQPPNLLEATKTLLMSTDPTAEQAESGENKSATDPLLWFKKLSNDTQDEDEEIDASTLKDPTRQLWRALDLSGQQLLHLSEKLFRYDFLTKLYLNGNGLTELPSSIRQLKSLTVLDVSQNLLSSFPPELGILFNLRYIYAFDNRLTDIPFEFGNLYELEFLGIEGNVNMNPEYVNILAKRGSRGLTIHLRDNAPRPTPPKSRQWIYFSNDGEIIEEQEYRQQQTEDDIVNTFTMMTYNTLCQHYATKKMYRYTPSWALDWDYRRERLKEQILDLQTDIICLQEVEHKTFDDFWQPIMLSHGYKGIFHVKSRAKTMKESSAYKVDGCATFYRTSKFQAVERKHFEYGRIAMSQDKFKKTEDLFNRFLNKDNIASVLILEHIPSGNKLVVANTHLHWDPEFNDVKTMQVGVLLDELQAVIRKHLSPKDITKVPLLICGDFNSKVHSAVYQLFSQGTVDKHEDIIGRDYGKFTEEGFRHPFHLQSSYDSIGELPYTNVSPTFTDVIDYIWYSTPSLSVKGVLGQVDPDYSKNIIGFPNADFPSDHIPLLSTFMFKKSSAPRPDTRVDFRSDFRGSRKT

SEQ ID NO: 20 - protein, 1118 aa, Pichia pastoris
  peptide (1)..(1118): Pan2
MEGWNEINRVPCMRPSFRISDPTRQSVPSALLFDDSHDLTWIGSEDGFVKSLADQMLTPYTSFRCHSSKVLQLLNNKRGILSLSENSIKLTSRTGLCRMNLHDISVNHSRSMAYTSNTENEVLLGGFQGKLVKLNLMRGEISDTIPYDAPVYTMSANLSQICLGRVDGTVDILDPRSNDIIASFSGHMRTLSSMDCRGNTLITTGYSVRNGTFYADPIANLYDLRTKSLMPPVTFPAGASFVKLHPKLPNVAILASSAGLIHFVNLYSPMNVSLYQADVGSYMNNFEVSPSGDFMGFTDSFQNVHLWSNAPDLSSAHIPNLASSLEQATMLPPPANSDIIPADSDSTVPLSSIGMPYYDQPLLSNWPFDMKFSLGHVPKKINPELINSSKSVVENYNAHAFGQNNLPSGMGVNMNIPGVFKTDTDTRIAVYSREKYGPRNVAQPYYRLHDRSNQKNKFVPKFISERLDVDEKSSSTAENTNSDFKDTLYEQLFDCKPHSSQEVPYCFTKLDIHYSKFGVHDFDFEFFNKTNLSGLESHVQSAYCNSLLQLYRFAPLMFNYAVGSLAEESVDNNSLLTELGFLFDMMVKASGQHIAPSNFQKMLSKIPEASRILDIYNNGYNRDEHSQRVLIQSFNNFLLERISLDEMNQAKSNAPHIFNTIMGIPVECEFIGVGCGLRKIGNTTLYSLDVRHPKTNNVVLNKKLNHSIIPYIELALSRNLSVNMACDNCNETHAFDTLYTVKDLPPILSLNLELTNQELSELKASKTNWLASEFYASMNKGRICLKSITTGFRHLKYELLGYVAQVTDKEGNSNLVTFVKVGKDEWFLFNDFLVMPISEHEVLNLNYWWKKPVIVIYKNSESTNMFDYEGWRQNLNQDILYRDHFSRGTREGKIIEYELLTKEEAPQPGTLVAIDAEFVVIEPELVEFNSDGTKKVIRPLKNSLARVSVLRGDTGPKEGIPFIDDYVIIEEPINDYLTSWSGIEPDDLNLEKSKRSLTTLQAVYRKLWLLLNLGCIFVGHGLINDFRTINLSVPKQQVRDTAELYFLKKEKRKLSLKFLTYAVLRREVQKGNHDSIEDAKAALMLYRKYIQLNNTGELQHTLEEVYMEGQMLSFKVPT

SEQ ID NO: 21 - protein, 614 aa, Pichia pastoris
  peptide (1)..(614): Pan3
MSANSNSTNTENHDWAKDIDCKNVFIHGYCKFENKGCYFKHPSDDSIEKNKETKETKSSAPLSSGTEEGSSIRKKFSFEAPSFTPSGSVASLTNKFSTMSPKLDEIPTFVPTSQQTPKKPQQVSAESNDYFAPPNSMFTPSVPSDNSQPGLPLSAQFPQHGTTAPHNVVPEMFYAQATQYPLNYNLYAPPPPPHFNLHLNPNERTVSSFFIDDNLRESLQKRNEACLQTFSNPSIPDIVGVYHSLVPLNNNFDNNSARYGAVSHMYKATSNKDARLYALRRIENVNITDKQAFKTIKAWSSIENSNIVKVHEAFTTTVFGGNSLVVAYDFYGNAKTLIEIHFQNQPELITEQHLWSYLIQLVNALNEVHDKGLAVRSIDLSKVIVTNKNRIKLSGCGIVDILQHENTEDIAQLQKKDLELLAKLLYDLSITSIYGSVSFDDKTQDQIIDHLKFSDDYKATLKYLISADFNLKEIQTRIAPRLLDVIDGLQNSNDFIESQLSTELENARLVRLMSKLSFLHGRPEHEGDPNWSESSANYPLTLFVDYVYHQVDERGYPVVDLAHVITCLNKLDAGIEERILLVSKDEKNCIIISYKELKTLIEEGFNELRMRKDI

SEQ ID NO: 22 - protein, 186 aa, Pichia pastoris
  peptide (1)..(186): Rpb4
MNVSTSTVGARRRRAKQQVDDEENATLLRLGPEFALKQYDHDGNEHDLIALSLSESRLLIREALKARSRARNGGVDIESSNGEIDDDELAKVTSGAVANGVVKKTLDYLNTFARFKDEETCTAVDQLLHNSSDCSVLHPFEIAQLSSLGCEDVDEAITLIPSLAAKKEVNLQRILDELNRLEDPYK

SEQ ID NO: 23 - protein, 171 aa, Pichia pastoris
  peptide (1)..(171): Rpb7
MFFLKDLSLILTLHPSYFGPQMNQYLREKLLTDVEGTCTGQFGYIVTVLDGMNIDVGKGRIIPGSGSAEFEVKYRAVVWKPFKGEVVDAIVSNVSPIGFFADVGPLNVFVSTRLIPDNLVYNPSNSPPAYMSNDELITKGSKVRLKVVGTRTDVNEIYAIGSIKEDFLGAI

SEQ ID NO: 24 - protein, 656 aa, Aspergillus niger
  peptide (1)..(656): CCR4
MNGGQAHQRFGMQIPKFQSQSHHPHPAQQAHHHAHHNQASHSINHQHNFSSGALAAATPHFTPGPLQNGAHVNVDEDIDETMNEHWQQQLQLAAESRQASSPHYYARTVAQQTKGIQIAPSQPESQENGSGDRNGLVKSKPAPRQGWHALDFGGQGLRALATSLFHYTFLEKLYLNHNKLKTLPPAIGQLRKLTHLDLSSNDISELPEEIGMLTSLKQLLLFDNNIRTLPFEMGYLYRLEMLGIEGNPLNDVLKSQIIKEGTKALVRYLREEMPVHLPPPDRDWIILDETASSSNSPTEKITVLSHNALCDSSATPSHFGYTPSRVLSWEFRRELILSELRSHDSDIICLQEIDQGSYNGFFREQLAYNDYKGVYWPRGRAMGMQEEEAKSVDGCATFFKGSKFILLDKQMINFGQTAVRRPDAKGQDDIYNRLWQKDHIAVVIFLENRLTGSRFIVVNAHLYWDPAFKDVKLIQTAILMEEITKLSEKYAKFPPCTDKTAFRFSEAEVEYASGDQIPLFMCGDFNSAPGSAAYNLVAHGRLTESHPDLEKRLYGNLSRVGMTHPFKLKSAYNSIGELSFTNYTPDFKDILDYIWYTSNTLHVSALLGEVDKEYLQKVPGFPNFHFPSDHVALFAEFTVKGKKGKVVEADFGPQRN

SEQ ID NO: 25 - protein, 1150 aa, Aspergillus niger
  peptide (1)..(1150): Pan2
MEADWDELSRIPVPAPSVHALPTIATAIAFDDVMELLWGRITSFFGPELQRYTSVRAHPATEPVRQIIFHDRGVISLSPKSVHMITRRGLTQWHIAHEEMTDLRCMSFTAQTNRIIVAGCQKSMFTIDIDKGIIIDKLHTEYNYTIMKKSRYLCAATDTGSVNALSLNDFSVVKSWKAHGTAVNDMDARNDLLVTCGFSVRHLGSPIVDPLANVYDLKTLSPLPPIPFHAGAAYVRMHPKLHTTSFVASQTGQLQVVDLMNPNAINLRQANVSFMLGIDLSPSGEALAINDAECAIHLWGSPAKVHFNEMSKEAEFGDVAPRPPTLDWSPETPLSMIGMPYYHERLFSAWPSHLVFEVGSPPPQVDQALIPYLRPAELGHHAPNPKKTRRYQVENTRALASAEPALIAPKFLSEKAREQNKAKSEGAISDAAEALAGAKINGETDDDPLLKYSNVEIKYSRFGVDDFDFRFYNQTTFSGLETHIANSFTNALLQLFKFIPYIRNVALHHAASSCIFETCLLCEMGYLFDMLEKASGQNCQATNLLKTFSSYREASNLGLFEENLTNKSLSAAIQAVNRFFLGQISHDFRMISPSSDDLDHRLATVASESIRCMFCQNEIVRPGNSLVNELNYPAIDIKQARRNPAFRFSNILRASIEREAQNRGWCNYCRRYQQVAIRKSVHRMPQVLMLNAALTNPICRRLWAIPGWLPEEVGIVIEGGQILCFEGEDLKLRVQAKMPGLVVYDLVGLVCEIDIPEHQKAHLVSFINVSISSREPETKNKWHLFNDFLVTEVDKEEALRFNQPWKIPCVLAYQVQDGRHAMDDTWKDALDTTLLFRDWSLNGGRPVESRVTLSEEEKPTPGTPVALDTEFVDLEKAEIDVKADGSQEIVRPSKSGLARVSVLRGSGIREGVPFIDDYITIKENIVDYVTQYSGIKPGDLDPRVSQHNLVPLKVAYKKLWLLLNLGCVFVGHGLASDFRKVNIQVPKSQTVDTQYLFFHPGKNRRLSLRYLAWAVFKEYIQEEPADDSQGHDSIEDARMALRLWKKFKEYEDAGIVSQILEEIFREGSKLGFRPPPRNGVPTVLSRPGTAVTMQNNSGRNTPSTSDVAGAAASAPATPRQAFRRSIALTPSNGSFAGPGTGDFFSGSPLK

SEQ ID NO: 26 - protein, 642 aa, Aspergillus niger
  peptide (1)..(642): Pan3
MATTGKSATLEDARHGTGSPKMKGRGCAFNHDPHKVNSGYQSDSNKKRLNVDSPSFTPSILSSNGSSPTSQSATMKKMATISPKAASAAPFQPRSISSRSNSSTPTTRPGTMTPDWSVAEVQEFVPQGFDTAHIGSLQGNGTAGVPSTSAFDPFVTAPNPLSAANAVGPVQANPFSHDTAAALNGAAFFANQSGFQQPVQYHMYAPIGPHSQNTLGYQRNVHDLFLPNDLREEMQKKAAATLQTLPNTQLPAQVDYFHSLVPLDLNHQKNATIFGFPSWVYKAQSSKDGNFYALRRLEGFRLTNEKAIRSVQAWKRVCNGSVVTVHDAFTSRSFQDSSLIFVTDYHPLSKTLAEQHLGAGNRFQGRHNTHIPEQVLWGYMTQIANALKAIHASQLAARIIDPSKILLTGRNRIRLNACAIMDVVQFDTQRSLAELQRQDLVNFGQLIVTLGANQPNVMHNPTKAMEHFTRAYTAQLKNSVFWLLNGLQKDQERNIDIFITGISSTLMSTFDSALHLDDQLTSDLSRELENGRLVRLMTKLNFVNERPEYEHDRQWSENGERYFLKIFRDYVFHQVDAQGDPVVDLGHVLMCLNKLDAGTDEKITLISRDEQSCFVVSYKELKKALESSFQALLKPSASRRLH

SEQ ID NO: 27 - protein, 148 aa, Aspergillus niger
  peptide (1)..(148): Rpb4
MSVQLPPATHRKRTLPQGELEAASTLKLGADQNTHTLSLSEARLVINKVLENKRRGGKKYEEPENLTKTLDYLEVFARFKDEENIKAVERLLNSHTELEMFERSQLGSLCCDNAEEAKSLIPSLQHKISDGDLQELLDELTKLRNFTE

SEQ ID NO: 28 - protein, 167 aa, Aspergillus niger
  peptide (1)..(167): Rpb7
MFFLKEEVKVITLHPSYFGPNMREYLINRLNEEEEGRCTGDHFVICVMDMVDIGEGRVLPGSGQAEYTIKYRAIIWKPFRGETVDAIVTSVKPTGIFTLAGPLSVFIARKNIPSDIKWEPNTVPPQYTDHADQVIEKGTSLRLKILGVKPDVAAINAIGTIKEDYLG

SEQ ID NO: 29 - DNA, 4886 bp, artificial sequence
  synthetic
  misc_feature (1)..(4886): Plasmid pLysS
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggcccattggctgcctcccacacttggatatgcctcctcggagccttatagaattgtttataagacttgcgcattatttgacctccaatgcgaacaaagggaaaccgctgtggtctccctttagtgagttcaattaattatccacggtcagaagtgaccagttcgttcttctcccaccaacgcttaaggtcgaacgaagggcaagccttcggcgccacctcatgatgggcgcgaagaccagcgccttcgtacttagccagcagtgtgacaagcagtgagcgaagggattgcatttgggctggcgtaaagttagcgtcgaacttacctttatcgtcgataccaccaacaaggcagacgccgatagagttgtggttgtaacccttagcgtgagagcctacagccatctcatctcgtcctgcctccacagtaccgtctcgcttgatgataaagtggtatcccacatcgagccaaccctgctctttgtgccactggcgaatctcacggacaccaacattctgacttggcttggtagccgagcagtgaacaaagattgcgtcagtagattcacgttgtttaaactgtacacgagccattatttctttcctcctttcctttttaatctatcaaaggggacccggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 30 - DNA, 660 bp, artificial sequence
  synthetic
  CDS (1)..(660): Gene encoding chloramphenicol acetyltransferase, which confers resistance to chloramphenicol
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 31 - protein, 219 aa, artificial sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 32 - DNA, 103 bp, artificial sequence
  synthetic
  promoter (1)..(103): cat promoter
tgatcggcacgtaagaggttccaactttcaccataatgaaataagatcactaccgggcgtattttttgagttatcgagattttcaggagctaaggaagctaaa

SEQ ID NO: 33 - DNA, 545 bp, Artificial sequence
  synthetic
  rep_origin (1)..(545): P15A ori
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaa

SEQ ID NO: 34 - DNA, 34 bp, Artificial sequence
  synthetic
  promoter (1)..(34): Phi 3.8 promoter
taattaattgaactcactaaagggagaccacagc

SEQ ID NO: 35 - DNA, 456 bp, artificial sequence
  synthetic
  CDS (1)..(456): T7 lysozyme
atggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataa

SEQ ID NO: 36 - protein, 151 aa, artificial sequence
  Synthetic Construct
MARVQFKQRESTDAIFVHCSATKPSQNVGVREIRQWHKEQGWLDVGYHFIIKRDGTVEAGRDEMAVGSHAKGYNHNSIGVCLVGGIDDKGKFDANFTPAQMQSLRSLLVTLLAKYEGAVLRAHHEVAPKACPSFDLKRWWEKNELVTSDRG

SEQ ID NO: 37 - DNA, 29 bp, Artificial sequence
  synthetic
  promoter (1)..(29): tet promoter
ttgacagcttatcatcgataagctttaat

SEQ ID NO: 38 - DNA, 8447 bp, Artificial sequence
  synthetic
  misc_feature (1)..(8447): Plasmid pMax
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaaaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 39 - DNA, 3186 bp, E. coli
  CDS (1)..(3186): rne(D346N) encoding mutant RNase E of E.coli comprising a single point mutation D346N
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaa

SEQ ID NO: 40 - protein, 1061 aa, E. coli
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVINFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKSAPATPAAPAQPGLLSRFFGALKALFSGGEETKPSEQPTPKAEAKPERQQDRRKPRQNNRRDRNERRDTRSERTEGSDNREENRRNRRQAQQQTAETRESRQQVEVTEKARTTDEQQAPRRERSRRRNDDKRQAQQEAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 41 - DNA, 119 bp, Artificial sequence
  synthetic
  promoter (1)..(119): rhaBAD promoter
caccacaattcagcaaattgtgaacatcatcacgttcatctttccctggttgccaatggcccattttcctgtcagtaacgagaaggtcgcgaattcaggcgctttttagactggtcgta

SEQ ID NO: 42 - DNA, 54 bp, Artificial sequence
  synthetic
  misc_feature (1)..(53): BCD
atgaaagcaattttcgtactgaaacatcttaatcatgctaaggaggttttctaa

SEQ ID NO: 43 - DNA, 41 bp, Artificial sequence
  synthetic
  terminator (1)..(41): rrnc terminator
agaaatcatccttagcgaaagctaaggattttttttatctg

SEQ ID NO: 44 - DNA, 48 bp, Artificial sequence
  synthetic
  terminator (1)..(48): T7 terminator
ctagcataaccccttggggcctctaaacgggtcttgaggggttttttg

SEQ ID NO: 45 - DNA, 7855 bp, Artificial sequence
  synthetic
  misc_feature (1)..(7855): Plasmid pMax-noLysS
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaaaggttacctagaaatcatccttagcgaaagctaaggattttttttatctgggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 46 - DNA, 5144 bp, Artificial sequence
  synthetic
  misc_feature (1)..(5144): Plasmid pET28a+
taatacgactcactataggggaattgtgagcggataacaattcccctctagaaataattttgtttaactttaagaaggagaccagtttctagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 47 - DNA, 19 bp, Artificial sequence
  synthetic
  promoter (1)..(19): T7 promoter
taatacgactcactatagg

SEQ ID NO: 48 - DNA, 25 bp, Artificial sequence
  synthetic
  misc_feature (1)..(25): lac operator
ggaattgtgagcggataacaattcc

SEQ ID NO: 49 - DNA, 456 bp, Artificial sequence
  synthetic
  rep_origin (1)..(456): f1 ori
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaattt

SEQ ID NO: 50 - DNA, 816 bp, Artificial sequence
  synthetic
  CDS (1)..(816): KanR
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 51 - protein, 271 aa, Artificial sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 52 - DNA, 589 bp, Artificial sequence
  synthetic
  rep_origin (1)..(589): pBR322_ori
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 53 - DNA, 143 bp, Artificial sequence
  synthetic
  misc_feature (1)..(143): bom
cctgatgcggtattttctccttacgcatctgtgcggtatttcacaccgcatatatggtgcactctcagtacaatctgctctgatgccgcatagttaagccagtatacactccgctatcgctacgtgactgggtcatggctgcg

SEQ ID NO: 54 - DNA, 192 bp, Artificial sequence
  synthetic
  misc_feature (1)..(192): rop
gtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctga

SEQ ID NO: 55 - DNA, 78 bp, Artificial sequence
  synthetic
  promoter (1)..(78): lacI promoter
gacaccatcgaatggcgcaaaacctttcgcggtatggcatgatagcgcccggaagagagtcaattcagggtggtgaat

SEQ ID NO: 56 - DNA, 1083 bp, Artificial sequence
  synthetic
  CDS (1)..(1083): lacI encoding lac repressor
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 57 - protein, 360 aa, Artificial sequence
  Synthetic Construct
VKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 58 - DNA, 7772 bp, Artificial sequence
  synthetic
  misc_feature (1)..(7772): Plasmid pP450
taatacgactcactataggggaattgtgagcggataacaattcccctctagaaataattttgtttaactttaagaaggagactcgaccatggaattatcacaagtttgtacaaaaaaggaggctggcgccggaaccaattcagtcgactggatccaagaaggagatataaccccctcagcagcgatggcgacaatggaggtagaggccgcggccgccacggtgctggccgcgcccttgctgtcctcctccgcgatcctcaaactgctgctattcgtagtgacgctctcgtacctggcccgagccctgaggcggccacgcaaaagcaccaccaagtgcagcagcacaacgtgcgcctcgcccccggccggcgttggcaacccgccgctcccaccgggtcccgtgccgtggcccgtcgtcggcaacctgccggagatgctgctgaacaagccggcattccgctggatccaccagatgatgcgcgagatgggcacggacatcgcctgcgtcaagcttggcggcgtccacgtcgtgtccatcacctgcccggagatcgcgcgggaggtgctccggaagcaggacgccaacttcatatcccgcccgctcaccttcgcctccgagacgttcagcggcgggtaccggaacgccgtgctctcgccctacggcgaccagtggaagaagatgcgccgcgtcctcacctccgagatcatctgcccgtcccgccacgcctggctccacgacaagcgcaccgacgaggccgacaacctcacccgctacgtctacaacctcgccaccaaagccgccaccggcgacgtcgccgtcgacgtcaggcacgtcgctcgtcactattgcggcaacgttatccgccgcctcatgttcaacaggcgctacttcggcgagccccaggctgacggcggtccggggccgatggaggtgctgcatatggacgccgtgttcacctccctcggcctcctctacgccttctgcgtctccgactacctcccctggctgcggggcctcgacctcgacggccacgagaagatcgtcaaggaggctaacgtggcggtgaacaggctccacgacacggtcatcgacgaccggtggaggcagtggaagagcggcgagcggcaggagatggaggacttcctggatgtgctcatcactctcaaggacgcccagggcaacccgctgctgaccatcgaggaggtcaaagcgcagtcacaggacatcacgttcgcggcggtggacaacccgtcgaacgccgtggagtgggcgctggcagagatggtgaacaacccggaggtgatggcgaaggcgatggaggagctggaccgcgtcgtcggacgggagaggctagtgcaggagtcggacattccgaagctcaactacgtgaaggcctgcatccgggaggctttccgtctgcacccggtggcgcccttcaacgtgccccacgtcgcgctcgccgacaccaccatcgccggctaccgcgttcccaagggcagccacgtgatcctgagccgcacggggctgggccgcaacccgcgcgtgtgggacgagcccctgcgcttctacccggaccgacacctcgccaccgccgcgtccgacgtcgcgctcaccgagaacgacctgcggttcatctccttcagcaccggccgccgcggctgcatcgccgcgtcgctcggcaccgccatgagcgtcatgctcttcggaaggctcctgcaggggttcacctggagcaagcccgccggggtggaggccgtggacctcagcgagtccaagagcgacaccttcatggccaccccgctggtgctgcacgctgagcccaggctgccggcgcacctctacccgtccatctccatcggatcgccagctgagggagaaaacctgtacttccagggtcaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaaaagcttgcggcccatcatcatcaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 59 - DNA, 2469 bp, Artificial sequence
  synthetic
  CDS (1)..(2469): Membrane protein P450(SbCYP79A1)-GFP-His8
atggcgacaatggaggtagaggccgcggccgccacggtgctggccgcgcccttgctgtcctcctccgcgatcctcaaactgctgctattcgtagtgacgctctcgtacctggcccgagccctgaggcggccacgcaaaagcaccaccaagtgcagcagcacaacgtgcgcctcgcccccggccggcgttggcaacccgccgctcccaccgggtcccgtgccgtggcccgtcgtcggcaacctgccggagatgctgctgaacaagccggcattccgctggatccaccagatgatgcgcgagatgggcacggacatcgcctgcgtcaagcttggcggcgtccacgtcgtgtccatcacctgcccggagatcgcgcgggaggtgctccggaagcaggacgccaacttcatatcccgcccgctcaccttcgcctccgagacgttcagcggcgggtaccggaacgccgtgctctcgccctacggcgaccagtggaagaagatgcgccgcgtcctcacctccgagatcatctgcccgtcccgccacgcctggctccacgacaagcgcaccgacgaggccgacaacctcacccgctacgtctacaacctcgccaccaaagccgccaccggcgacgtcgccgtcgacgtcaggcacgtcgctcgtcactattgcggcaacgttatccgccgcctcatgttcaacaggcgctacttcggcgagccccaggctgacggcggtccggggccgatggaggtgctgcatatggacgccgtgttcacctccctcggcctcctctacgccttctgcgtctccgactacctcccctggctgcggggcctcgacctcgacggccacgagaagatcgtcaaggaggctaacgtggcggtgaacaggctccacgacacggtcatcgacgaccggtggaggcagtggaagagcggcgagcggcaggagatggaggacttcctggatgtgctcatcactctcaaggacgcccagggcaacccgctgctgaccatcgaggaggtcaaagcgcagtcacaggacatcacgttcgcggcggtggacaacccgtcgaacgccgtggagtgggcgctggcagagatggtgaacaacccggaggtgatggcgaaggcgatggaggagctggaccgcgtcgtcggacgggagaggctagtgcaggagtcggacattccgaagctcaactacgtgaaggcctgcatccgggaggctttccgtctgcacccggtggcgcccttcaacgtgccccacgtcgcgctcgccgacaccaccatcgccggctaccgcgttcccaagggcagccacgtgatcctgagccgcacggggctgggccgcaacccgcgcgtgtgggacgagcccctgcgcttctacccggaccgacacctcgccaccgccgcgtccgacgtcgcgctcaccgagaacgacctgcggttcatctccttcagcaccggccgccgcggctgcatcgccgcgtcgctcggcaccgccatgagcgtcatgctcttcggaaggctcctgcaggggttcacctggagcaagcccgccggggtggaggccgtggacctcagcgagtccaagagcgacaccttcatggccaccccgctggtgctgcacgctgagcccaggctgccggcgcacctctacccgtccatctccatcggatcgccagctgagggagaaaacctgtacttccagggtcaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaaaagcttgcggcccatcatcatcaccaccaccaccactga

SEQ ID NO: 60 - protein, 822 aa, Artificial sequence
  Synthetic Construct
MATMEVEAAAATVLAAPLLSSSAILKLLLFVVTLSYLARALRRPRKSTTKCSSTTCASPPAGVGNPPLPPGPVPWPVVGNLPEMLLNKPAFRWIHQMMREMGTDIACVKLGGVHVVSITCPEIAREVLRKQDANFISRPLTFASETFSGGYRNAVLSPYGDQWKKMRRVLTSEIICPSRHAWLHDKRTDEADNLTRYVYNLATKAATGDVAVDVRHVARHYCGNVIRRLMFNRRYFGEPQADGGPGPMEVLHMDAVFTSLGLLYAFCVSDYLPWLRGLDLDGHEKIVKEANVAVNRLHDTVIDDRWRQWKSGERQEMEDFLDVLITLKDAQGNPLLTIEEVKAQSQDITFAAVDNPSNAVEWALAEMVNNPEVMAKAMEELDRVVGRERLVQESDIPKLNYVKACIREAFRLHPVAPFNVPHVALADTTIAGYRVPKGSHVILSRTGLGRNPRVWDEPLRFYPDRHLATAASDVALTENDLRFISFSTGRRGCIAASLGTAMSVMLFGRLLQGFTWSKPAGVEAVDLSESKSDTFMATPLVLHAEPRLPAHLYPSISIGSPAEGENLYFQGQFSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKKLAAHHHHHHHH

SEQ ID NO: 61 - DNA, 6878 bp, Artificial sequence
  synthetic
  misc_feature (1)..(6878): Plasmid pHtpX
taatacgactcactataggggaattgtgagcggataacaattcccctctagaaataattttgtttaactttaagaaggagaccagtttatgatgcgaatcgcgctcttcctgctaacgaacctggccgtaatggtcgttttcgggctggtactgagcctgacagggatacagtcgagcagcgttcaggggctgatgatcatggccttgctgttcggttttggtggttccttcgtttcgcttctgatgtccaaatggatggcattacgatctgttggcggggaagtgatcgagcaaccgcgtaacgaaagggaacgttggctggtcaatactgtagcaacccaggctcgtcaggcggggatcgctatgccgcaagtggctatctaccatgcgccggacatcaacgcttttgcaaccggtgcgcgccgtgatgcctctctggttgctgtcagcaccggtttgctgcagaacatgagcccggatgaagccgaggcggtaattgctcacgaaatcagccacatcgccaatggtgatatggtcaccatgacgctgattcagggcgtggtgaacaccttcgttatctttatttcccgtattctggcgcagcttgccgcgggttttatgggcggaaatcgtgatgaaggtgaagagagcaacggcaacccgctgatctactttgcggttgcaacggttctggaactggtgtttggtattctggcgagcattatcaccatgtggttctcgcgtcatcgtgaattccatgctgatgccggttcggcaaaactggttggtcgcgagaaaatgattgccgcgctgcagcgcctgaaaaccagctatgaaccgcaagaagcaaccagcatgatggctctctgcattaacggtaagtcgaaatcgctcagtgagttgttcatgacccacccgccgctggataaacgaattgaagctctgcgtacgggtgaatacctgaagtcggtacctggatccgaaaacctgtacttccagggtcaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaaaagcttgcggcccatcatcatcaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 62 - DNA, 1671 bp, Artificial sequence
  synthetic
  CDS (1)..(1671): Membrane protein HtpX-GFP-His8
atgatgcgaatcgcgctcttcctgctaacgaacctggccgtaatggtcgttttcgggctggtactgagcctgacagggatacagtcgagcagcgttcaggggctgatgatcatggccttgctgttcggttttggtggttccttcgtttcgcttctgatgtccaaatggatggcattacgatctgttggcggggaagtgatcgagcaaccgcgtaacgaaagggaacgttggctggtcaatactgtagcaacccaggctcgtcaggcggggatcgctatgccgcaagtggctatctaccatgcgccggacatcaacgcttttgcaaccggtgcgcgccgtgatgcctctctggttgctgtcagcaccggtttgctgcagaacatgagcccggatgaagccgaggcggtaattgctcacgaaatcagccacatcgccaatggtgatatggtcaccatgacgctgattcagggcgtggtgaacaccttcgttatctttatttcccgtattctggcgcagcttgccgcgggttttatgggcggaaatcgtgatgaaggtgaagagagcaacggcaacccgctgatctactttgcggttgcaacggttctggaactggtgtttggtattctggcgagcattatcaccatgtggttctcgcgtcatcgtgaattccatgctgatgccggttcggcaaaactggttggtcgcgagaaaatgattgccgcgctgcagcgcctgaaaaccagctatgaaccgcaagaagcaaccagcatgatggctctctgcattaacggtaagtcgaaatcgctcagtgagttgttcatgacccacccgccgctggataaacgaattgaagctctgcgtacgggtgaatacctgaagtcggtacctggatccgaaaacctgtacttccagggtcaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaaaagcttgcggcccatcatcatcaccaccaccaccactga

SEQ ID NO: 63 - protein, 556 aa, Artificial sequence
  Synthetic Construct
MMRIALFLLTNLAVMVVFGLVLSLTGIQSSSVQGLMIMALLFGFGGSFVSLLMSKWMALRSVGGEVIEQPRNERERWLVNTVATQARQAGIAMPQVAIYHAPDINAFATGARRDASLVAVSTGLLQNMSPDEAEAVIAHEISHIANGDMVTMTLIQGVVNTFVIFISRILAQLAAGFMGGNRDEGEESNGNPLIYFAVATVLELVFGILASIITMWFSRHREFHADAGSAKLVGREKMIAALQRLKTSYEPQEATSMMALCINGKSKSLSELFMTHPPLDKRIEALRTGEYLKSVPGSENLYFQGQFSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKKLAAHHHHHHHH

SEQ ID NO: 64 - DNA, 7658 bp, Artificial sequence
  synthetic
  misc_feature (1)..(7658): Plasmid pYqiK
taatacgactcactataggggaattgtgagcggataacaattcccctctagaaataattttgtttaactttaagaaggagaccagtttatggatgatattgttaattctgtgccctcctggatgtttaccgcaattattgccgtatgcattctgtttattattggaattattttcgccaggctctatcgtcgcgcttcggcagagcaagcttttgttcgtactggtttaggtgggcaaaaagtggtaatgagcggtggcgcaatcgtgatgccgatctttcatgaaataatccccatcaatatgaatactctgaagctggaagtcagccgctcaaccattgatagcctgattacgaaagatcgtatgcgcgtcgatgtagtagtcgctttctttgtgcgggtaaaaccttcagtagaagggattgccaccgctgcccagacgctggggcaacgcaccctgtcgcctgaagacttacgtatgttggttgaagataaatttgtcgatgccctccgtgcaacagctgcgcaaatgaccatgcatgagttacaggatacccgcgagaactttgtgcagggggtgcaaaatacagtggcagaagacctgtcgaaaaacggtctggaactggagagcgtttcacttaccaactttaaccagacctcgaaagaacatttcaatccgaacaatgcctttgacgccgaaggtttaaccaaactgactcaggaaacagagcgccgtcgccgcgaacgtaacgaagttgaacaggatgtagaagttgcggtgcgtgaaaaaaatcgcgatgcgctatcgcgcaagctggagattgaacagcaagaagcgtttatgacgcttgagcaggagcagcaggttaaaacccgtactgccgaacagaatgcacgtattgcggcttttgaagctgaacgtcgtcgtgaagcagagcagacacgaattctggctgaacgacagattcaggaaacagaaatcgaccgcgaacaggccgtccgctcaagaaaggttgaagctgaacgtgaagttcgcattaaagagatcgaacagcagcaggtcaccgaaatcgctaaccagacgaaatcgatcgctattgccgccaaatcggaacaacagtcccaggcagaagcgcgtgctaatctcgcacttgcagaagcggtaagcgcccaacaaaacgtagaaaccactcgccagactgccgaagccgatcgtgctaaacaagttgccctaatcgctgccgcgcaggatgcagaaaccaaagcggttgaactgaccgtgcgggcgaaagcagaaaaagaagccgcagagatgcaggcggcggctatcgttgagttagccgaagctacacgtaaaaagggtctggcggaagcagaagcacaacgtgcgctgaacgatgctatcaacgtactttctgatgaacaaaccagccttaaattcaaactggccttgttgcaggcgctgcctgcggtaatagagaaatccgttgagccgatgaagtcaatcgacggtatcaagattattcaggtcgatggtctgaatcgtggcggcgctgcgggtgatgcaaacacgggtaatgtggggggcggaaacctggcggaacaagcattatcagccgctctctcttaccgcacacaggcaccgctgattgactccttgctcaatgaaattggcgtttcaggcggctcactggcggcattgacttcacccttaacctcaacaactcccgtcgaagaaaaagcagagtcggtacctggatccgaaaacctgtacttccagggtcaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaaaagcttgcggcccatcatcatcaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 65 - DNA, 2451 bp, Artificial sequence
  synthetic
  CDS (1)..(2451): Membrane protein YqiK-GFP-His8
atggatgatattgttaattctgtgccctcctggatgtttaccgcaattattgccgtatgcattctgtttattattggaattattttcgccaggctctatcgtcgcgcttcggcagagcaagcttttgttcgtactggtttaggtgggcaaaaagtggtaatgagcggtggcgcaatcgtgatgccgatctttcatgaaataatccccatcaatatgaatactctgaagctggaagtcagccgctcaaccattgatagcctgattacgaaagatcgtatgcgcgtcgatgtagtagtcgctttctttgtgcgggtaaaaccttcagtagaagggattgccaccgctgcccagacgctggggcaacgcaccctgtcgcctgaagacttacgtatgttggttgaagataaatttgtcgatgccctccgtgcaacagctgcgcaaatgaccatgcatgagttacaggatacccgcgagaactttgtgcagggggtgcaaaatacagtggcagaagacctgtcgaaaaacggtctggaactggagagcgtttcacttaccaactttaaccagacctcgaaagaacatttcaatccgaacaatgcctttgacgccgaaggtttaaccaaactgactcaggaaacagagcgccgtcgccgcgaacgtaacgaagttgaacaggatgtagaagttgcggtgcgtgaaaaaaatcgcgatgcgctatcgcgcaagctggagattgaacagcaagaagcgtttatgacgcttgagcaggagcagcaggttaaaacccgtactgccgaacagaatgcacgtattgcggcttttgaagctgaacgtcgtcgtgaagcagagcagacacgaattctggctgaacgacagattcaggaaacagaaatcgaccgcgaacaggccgtccgctcaagaaaggttgaagctgaacgtgaagttcgcattaaagagatcgaacagcagcaggtcaccgaaatcgctaaccagacgaaatcgatcgctattgccgccaaatcggaacaacagtcccaggcagaagcgcgtgctaatctcgcacttgcagaagcggtaagcgcccaacaaaacgtagaaaccactcgccagactgccgaagccgatcgtgctaaacaagttgccctaatcgctgccgcgcaggatgcagaaaccaaagcggttgaactgaccgtgcgggcgaaagcagaaaaagaagccgcagagatgcaggcggcggctatcgttgagttagccgaagctacacgtaaaaagggtctggcggaagcagaagcacaacgtgcgctgaacgatgctatcaacgtactttctgatgaacaaaccagccttaaattcaaactggccttgttgcaggcgctgcctgcggtaatagagaaatccgttgagccgatgaagtcaatcgacggtatcaagattattcaggtcgatggtctgaatcgtggcggcgctgcgggtgatgcaaacacgggtaatgtggggggcggaaacctggcggaacaagcattatcagccgctctctcttaccgcacacaggcaccgctgattgactccttgctcaatgaaattggcgtttcaggcggctcactggcggcattgacttcacccttaacctcaacaactcccgtcgaagaaaaagcagagtcggtacctggatccgaaaacctgtacttccagggtcaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaaaagcttgcggcccatcatcatcaccaccaccaccactga

SEQ ID NO: 66 - protein, 816 aa, Artificial sequence
  Synthetic Construct
MDDIVNSVPSWMFTAIIAVCILFIIGIIFARLYRRASAEQAFVRTGLGGQKVVMSGGAIVMPIFHEIIPINMNTLKLEVSRSTIDSLITKDRMRVDVVVAFFVRVKPSVEGIATAAQTLGQRTLSPEDLRMLVEDKFVDALRATAAQMTMHELQDTRENFVQGVQNTVAEDLSKNGLELESVSLTNFNQTSKEHFNPNNAFDAEGLTKLTQETERRRRERNEVEQDVEVAVREKNRDALSRKLEIEQQEAFMTLEQEQQVKTRTAEQNARIAAFEAERRREAEQTRILAERQIQETEIDREQAVRSRKVEAEREVRIKEIEQQQVTEIANQTKSIAIAAKSEQQSQAEARANLALAEAVSAQQNVETTRQTAEADRAKQVALIAAAQDAETKAVELTVRAKAEKEAAEMQAAAIVELAEATRKKGLAEAEAQRALNDAINVLSDEQTSLKFKLALLQALPAVIEKSVEPMKSIDGIKIIQVDGLNRGGAAGDANTGNVGGGNLAEQALSAALSYRTQAPLIDSLLNEIGVSGGSLAALTSPLTSTTPVEEKAESVPGSENLYFQGQFSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKKLAAHHHHHHHH

SEQ ID NO: 67 - DNA, 8471 bp, Artificial sequence
  synthetic
  misc_feature (1)..(8471): Plasmid pYidC
taatacgactcactataggggaattgtgagcggataacaattcccctctagaaataattttgtttaactttaagaaggagaccagtttatggattcgcaacgcaatcttttagtcatcgctttgctgttcgtgtctttcatgatctggcaagcctgggagcaggataaaaacccgcaacctcaggcccaacagaccacgcagacaacgaccaccgcagcgggtagcgccgccgaccagggcgtaccggccagtggccaggggaaactgatctcggttaagaccgacgtgcttgatctgaccatcaacacccgtggtggtgatgttgagcaagctctgctgcctgcttacccgaaagagctgaactctacccagccgttccagctgttggaaacttcaccgcagtttatttatcaggcacagagcggtctgaccggtcgtgatggcccggataacccggctaacggcccgcgtccgctgtataacgttgaaaaagacgcttatgtgctggctgaaggtcaaaacgaactgcaggtgccgatgacgtataccgacgcggcaggcaacacgtttaccaaaacgtttgtcctgaaacgtggtgattacgctgtcaacgtcaactacaacgtgcagaacgctggcgagaaaccgctggaaatctcctcgtttggtcagttgaagcaatccatcactctgccaccgcatctcgataccggaagcagcaacttcgcactgcacaccttccgtggcgcggcgtactccacgcctgacgagaagtatgagaaatacaagttcgataccattgccgataacgaaaacctgaacatctcttcgaaaggtggttgggtggcgatgctgcaacagtatttcgcgacggcgtggatcccgcataacgacggtaccaacaacttctataccgctaatctgggtaacggcatcgccgctatcggctataaatctcagccggtactggttcagcctggtcagactggcgcgatgaacagcaccctgtgggttggcccggaaatccaggacaaaatggcagctgttgctccgcacctggatctgaccgttgattacggttggttgtggttcatctctcagccgctgttcaaactgctgaaatggatccatagctttgtgggtaactggggcttctccattatcatcatcacctttatcgttcgtggcatcatgtacccgctgaccaaagcgcagtacacctccatggcgaagatgcgtatgttgcagccgaagattcaggcaatgcgtgagcgtctgggcgatgacaaacagcgtatcagccaggaaatgatggcgctgtacaaagctgagaaggttaacccgctgggcggctgcttcccgctgctgatccagatgccaatcttcctggcgttgtactacatgctgatgggttccgttgaactgcgtcaggcaccgtttgcactgtggatccacgacctgtcggcacaggacccgtactacatcctgccgatcctgatgggcgtaacgatgttcttcattcagaagatgtcgccgaccacagtgaccgacccgatgcagcagaagatcatgacctttatgccggtcatcttcaccgtgttcttcctgtggttcccgtcaggtctggtgctgtactatatcgtcagcaacctggtaaccattattcagcagcagctgatttaccgtggtctggaaaaacgtggcctgcatagccgcgagaagaaaaaatccgaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaattgggtaccctcgagcaccaccaccaccaccactgacataggaggtcctcctatgtcaattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 68 - DNA, 2397 bp, Artificial sequence
  synthetic
  CDS (1)..(2397): Membrane protein YidC-GFP-His8
atggattcgcaacgcaatcttttagtcatcgctttgctgttcgtgtctttcatgatctggcaagcctgggagcaggataaaaacccgcaacctcaggcccaacagaccacgcagacaacgaccaccgcagcgggtagcgccgccgaccagggcgtaccggccagtggccaggggaaactgatctcggttaagaccgacgtgcttgatctgaccatcaacacccgtggtggtgatgttgagcaagctctgctgcctgcttacccgaaagagctgaactctacccagccgttccagctgttggaaacttcaccgcagtttatttatcaggcacagagcggtctgaccggtcgtgatggcccggataacccggctaacggcccgcgtccgctgtataacgttgaaaaagacgcttatgtgctggctgaaggtcaaaacgaactgcaggtgccgatgacgtataccgacgcggcaggcaacacgtttaccaaaacgtttgtcctgaaacgtggtgattacgctgtcaacgtcaactacaacgtgcagaacgctggcgagaaaccgctggaaatctcctcgtttggtcagttgaagcaatccatcactctgccaccgcatctcgataccggaagcagcaacttcgcactgcacaccttccgtggcgcggcgtactccacgcctgacgagaagtatgagaaatacaagttcgataccattgccgataacgaaaacctgaacatctcttcgaaaggtggttgggtggcgatgctgcaacagtatttcgcgacggcgtggatcccgcataacgacggtaccaacaacttctataccgctaatctgggtaacggcatcgccgctatcggctataaatctcagccggtactggttcagcctggtcagactggcgcgatgaacagcaccctgtgggttggcccggaaatccaggacaaaatggcagctgttgctccgcacctggatctgaccgttgattacggttggttgtggttcatctctcagccgctgttcaaactgctgaaatggatccatagctttgtgggtaactggggcttctccattatcatcatcacctttatcgttcgtggcatcatgtacccgctgaccaaagcgcagtacacctccatggcgaagatgcgtatgttgcagccgaagattcaggcaatgcgtgagcgtctgggcgatgacaaacagcgtatcagccaggaaatgatggcgctgtacaaagctgagaaggttaacccgctgggcggctgcttcccgctgctgatccagatgccaatcttcctggcgttgtactacatgctgatgggttccgttgaactgcgtcaggcaccgtttgcactgtggatccacgacctgtcggcacaggacccgtactacatcctgccgatcctgatgggcgtaacgatgttcttcattcagaagatgtcgccgaccacagtgaccgacccgatgcagcagaagatcatgacctttatgccggtcatcttcaccgtgttcttcctgtggttcccgtcaggtctggtgctgtactatatcgtcagcaacctggtaaccattattcagcagcagctgatttaccgtggtctggaaaaacgtggcctgcatagccgcgagaagaaaaaatccgaattcagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaattgggtaccctcgagcaccaccaccaccaccactga

SEQ ID NO: 69 - protein, 798 aa, Artificial sequence
  Synthetic Construct
MDSQRNLLVIALLFVSFMIWQAWEQDKNPQPQAQQTTQTTTTAAGSAADQGVPASGQGKLISVKTDVLDLTINTRGGDVEQALLPAYPKELNSTQPFQLLETSPQFIYQAQSGLTGRDGPDNPANGPRPLYNVEKDAYVLAEGQNELQVPMTYTDAAGNTFTKTFVLKRGDYAVNVNYNVQNAGEKPLEISSFGQLKQSITLPPHLDTGSSNFALHTFRGAAYSTPDEKYEKYKFDTIADNENLNISSKGGWVAMLQQYFATAWIPHNDGTNNFYTANLGNGIAAIGYKSQPVLVQPGQTGAMNSTLWVGPEIQDKMAAVAPHLDLTVDYGWLWFISQPLFKLLKWIHSFVGNWGFSIIIITFIVRGIMYPLTKAQYTSMAKMRMLQPKIQAMRERLGDDKQRISQEMMALYKAEKVNPLGGCFPLLIQMPIFLALYYMLMGSVELRQAPFALWIHDLSAQDPYYILPILMGVTMFFIQKMSPTTVTDPMQQKIMTFMPVIFTVFFLWFPSGLVLYYIVSNLVTIIQQQLIYRGLEKRGLHSREKKKSEFSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKLGTLEHHHHHH

SEQ ID NO: 70 - DNA, 25 bp, Artificial sequence
  synthetic
  misc_feature (1)..(25): hp6
tgacataggaggtcctcctatgtca

SEQ ID NO: 71 - DNA, 861 bp, Artificial sequence
  synthetic
  CDS (1)..(861): AmpR
atgtcaattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 72 - protein, 286 aa, Artificial sequence [sequence omitted]

SEQ ID NO: 73 - DNA, 6728 bp, Artificial sequence
  synthetic
  misc_feature (1)..(6728): Plasmid pMax-truncV489
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 74 - DNA, 1467 bp, Artificial sequence
  synthetic
  CDS (1)..(1467): rneD346N truncated at V489
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtg

SEQ ID NO: 75 - protein, 489 aa, Artificial sequence
  Synthetic Construct
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVINFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRV

SEQ ID NO: 76 - DNA, 6848 bp, Artificial sequence
  synthetic
  misc_feature (1)..(6848): Plasmid pMax-truncL529
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctgaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 77 - DNA, 1587 bp, Artificial sequence
  synthetic
  CDS (1)..(1587): rneD346N truncated at L529
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcaacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctg

SEQ ID NO: 78 - protein, 529 aa, Artificial sequence
  Synthetic Construct
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVINFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPAL

SEQ ID NO: 79 - DNA, 8447 bp, Artificial sequence
  synthetic
  misc_feature (1)..(8447): Plasmid pMax-D346X, wherein NNN may be TTT, CTG, AAT, GTG, AGC,CCG, ACC, GCG, TAT, CAT, CAG, AAA, GAA, TGC, or GGC
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcnnnttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaaaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 80 - protein, 1061 aa, E. coli
  PEPTIDE (1)..(1061): Translation of rneD346X, wherien X is Phe(F), Leu(L), Ile(I), Val(V), Ser(S), Pro(P), Thr(T), Ala(A), Tyr(Y), HIs(H), Gln(Q), Lys(K), Glu(E), Cyc(C), or Gly(G).
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIXFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKSAPATPAAPAQPGLLSRFFGALKALFSGGEETKPSEQPTPKAEAKPERQQDRRKPRQNNRRDRNERRDTRSERTEGSDNREENRRNRRQAQQQTAETRESRQQVEVTEKARTTDEQQAPRRERSRRRNDDKRQAQQEAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 81 - DNA, 8447 bp, Artificial sequence
  synthetic
  misc_feature (1)..(8447): pMax-rneWT
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaaaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 82 - DNA, 7112 bp, Artificial sequence
  synthetic
  misc_feature (1)..(7112): pMax-rneSTAR
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggaagaaaccaaaccgaccgagcaaccagcaccgaaagcagaagcgaaaccggaacgtcaacaggatcgtcgcaagcctcgtcagaacaaccgccgtgaaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 83 - DNA, 1851 bp, E. coli
  CDS (1)..(1851): rneSTAR encoding a truncated RNAse E
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggaagaaaccaaaccgaccgagcaaccagcaccgaaagcagaagcgaaaccggaacgtcaacaggatcgtcgcaagcctcgtcagaacaaccgccgtga

SEQ ID NO: 84 - protein, 616 aa, E. coli
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGRNQTDRATSTESRSETGTSTGSSQASSEQPP

SEQ ID NO: 85 - DNA, 9224 bp, Artificial sequence
  synthetic
  misc_feature (1)..(9224): Plasmid pMac-rneEvo21
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataaaccgctgagcaataactagcataaccccttggggcctctaaacgggtcttgaggggttttttgttaattaagcggccgcggcgcgcccgtcgacgcgcgcgcggctgaggatacctcagccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagactatatatgaaagcaattttcgtactgaaacatcttaatcatgctaaggaggttttctaatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaaggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaaaggttacctagaaatcatccttagcgaaagctaaggattttttttatctgtaattaattgaactcactaaagggagaccacagcggtttccctttgttcgcattggaggtcaaataatgcgcaagtcttataaacaattctataaggctccgaggaggcatatccaagtgtgggaggcagccaatgggccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 86 - DNA, 2187 bp, Artificial sequence
  synthetic
  CDS (1)..(2187): rneEvo21 encoding truncated RNase E
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaaggtaatgactccaacttattgatagtgttttatgttcagataatgcccgatgactttgtcatgcagctccaccgattttga

SEQ ID NO: 87 - protein, 728 aa, Artificial sequence
  Synthetic Construct
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKSAPATPAAPAQPGLLSRFFGALKALFSGGEETKPSEQPTPKAEAKPERQQDRRKPRQNNRRDRNERRDTRSERTEGSDNREENRRNRRQAQQQTAETRESRQQVEVTEKARTTDEQQAPRRERSRRRNDDKRQAQQEAKALNVEEQGNDSNLLIVFYVQIMPDDFVMQLHRF

SEQ ID NO: 88 - DNA, 295 bp, Artificial sequence
  synthetic
  promoter (1)..(295): araBAD promoter
aaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttg

SEQ ID NO: 89 - DNA, 54 bp, Artificial sequence
  synthetic
  promoter (1)..(54): Ptet promoter
ttgacaattaatcatccggctcgtataatggaattgtgagcggataacaattcc

SEQ ID NO: 90 - DNA, 82 bp, Artificial sequence
  synthetic
  promoter (1)..(82): Ptet promoter
tcatgtttgacagcttatcatcgattagctttaatgcggtagtttatcacagttaaattgctaacgcagtcaggcaccgtgt

SEQ ID NO: 91 - DNA, 29 bp, Artificial sequence
  synthetic
  promoter (1)..(29): Ptac promoter
ttgacaattaatcatcggctcgtataatg

SEQ ID NO: 92 - DNA, 51 bp, Artificial sequence
  synthetic
  promoter (1)..(51): PL promoter
ggcggtgttgacataaataccactggcggtgatactgagcacatcagcagg

(19) United States
(12) Patent Application Publication
(10) Pub. No.: US 2025/0263679 A1
(43) Pub. Date: Aug. 21, 2025

(54) MUTANT RNASE E FOR ENHANCING RECOMBINANT PROTEIN EXPRESSION

(71) Applicant: DANMARKS TEKNISKE UNIVERSITET, Kongens Lyngby (DK)

(72) Inventors: Morten Helge Hauberg NØRHOLM, Kongens Lyngby (DK); Sophia Andrea Helga HEYDE, kongens Lyngby (DK)

(21) Appl. No.: 18/563,026

(22) PCT Filed: Jun. 2, 2022
(86) PCT No.: PCT/EP2022/065066

§ 371 Date: Nov. 21, 2023

(30) Foreign Application Priority Data
Jun. 2, 2021 (EP) 21177466.6

(51) Int. Cl. C12N 9/22; C07K 14/245; C12N 9/12; C12N 9/80; C12N 15/70

(52) U.S. Cl. CPC C12N 9/22; C07K 14/245; C12N 9/1247; C12N 9/80; C12N 15/70; C12Y 207/07006; C12Y 301/26012; C12Y 305/01028; C07K 2319/60

(57) ABSTRACT

The invention provides a microbial host cell for enhanced recombinant expression of a target protein, said host cell comprising a mutant RNase E enzyme to be coexpressed with a target gene of interest. The invention further provides a method of enhancing recombinant protein expression using said microbial host cell. The method is particularly useful for the expression of proteins that are otherwise difficult to express in traditional expression systems, such as proteins which are toxic to the host cell. The invention further provides an auxiliary plasmid comprising a rne* gene encoding a mutant RNase E enzyme and a LysS gene encoding T7 lysozyme.

### FIELD OF THE INVENTION

The Invention provides a method of enhancing recombinant protein expression in microbial organisms by providing a mutant ribonuclease E (RNase E) gene, such as on an auxiliary plasmid, to be coexpressed with a target gene of interest. The method is particularly useful in the expression of proteins which are otherwise difficult to express in traditional expression systems.

### BACKGROUND

Recombinant production of proteins enables biochemical and structural studies, and the pET vectors hosted in Escherichia coli strain BL21(DE3) are the most popular approach in research laboratories. Fast growth, high cell density, inexpensive culturing, availability of a wide variety of pET expression vectors combined with the detailed knowledge of E. coli's genetics, physiology, and metabolism make it the preferred laboratory workhorse. However, the bacteria often show impaired growth and fitness loss when being used for protein production—a problem that is highly gene-specific, and clear guiding principles for gene and cell factory optimization are still lacking.

DE3 strains carry the T7 RNA polymerase (RNAP) gene under the control of the IacUV5 promoter, a stronger version of the native lacZ promoter inducible with IPTG. This allows the expression of any gene of interest under the control of a T7 promoter. Different strains have been isolated in which the toxicity of membrane protein production is reduced, leading to improved production yields: the “Walker strains” C41(DE3) and C43(DE3) (Miroux et al 1996), evolved in the late 1990s to tolerate over-production of membrane proteins, the more recently characterised derivative “mutant56” (Baumgarten et al 2017) evolved for higher production of the toxic membrane protein YidC, and the strains C44(DE3) and C45(DE3) (Angius et al 2018) evolved similarly. In all these cases, gene expression induced by IPTG inhibited colony formation on agar plates before mutations occurred and tolerance was achieved mainly due to reduced T7 RNAP activity, either via lacI mutations (Kwon et al 2015), or via promoter modifications, point mutations, or truncations in the T7 RNAP gene.

RNase E is an essential membrane-associated enzyme in E. coli Involved in the maturation of both ribosomal RNA and tRNA, as well as total mRNA decay, and mediates the assembly of a multi-enzyme complex referred to as the “RNA degradosome” (FIG. 1A). It has previously been shown that only the N-terminal half of RNase E (amino acids 1-529 in E. coli RNase E), accommodating the active catalytic domain, is essential for cell growth, and the C-terminal non-catalytic region is mostly disordered and known to function as a scaffold mediating the association of the enzymes polynucleotide phosphorylase (PNPase), ATP-dependent RNA helicase (Rhlb) and enolase.

E. coli RNase E (EcRNase E) is the founding member of Type I RNase Es, found in betaproteobacteria, gammaproteobacteria and cyanobacteria, and has been extensively characterised. It is a large protein containing 1061 amino acids and can be divided into two domains. The N-terminal domain (NTD) is responsible for the endoribonuclease activity and the C-terminal domain (CTD) forms the structural scaffold for an RNA-degrading multienzyme complex, the degradosome. The catalytic NTD consists of five subdomains (illustrated in FIG. 1B): an RNase H domain, an S1 domain, a 5′ sensor, a deoxyribonuclease (DNase) I domain and a small domain (Mardle et al 2019). It is a homotetramer formed by interactions between the small domains. The catalytic site is located in the DNase I domain and harbours a hydrated magnesium ion, coordinated by two aspartic acids, Asp₃₀₃, positioned by asparagine Asn₃₀₅, and Asp₃₄₆, that is essential for the hydrolytic cleavage of the RNA substrate. EcRNase E cleaves single-stranded A/U-rich regions and has a strong preference for a 5′ monophosphate. Specificity for uracil at the +2 position relative to the cleavage site is defined by the uracil pocket in the S1 domain. This pocket is comprised of Phe₆₇, positioned by Phe₅₇, and the Lys₁₁₂-Gly₁₁₃-Ala₁₁₄-Alanis₁₁₅ (KGAA) loop. In addition, recognition of a 5′ monophosphorylated substrate requires the 5′ sensor pocket, formed by amino acids Gly₁₂₄, Val₁₂₈, Arg₁₄₁, Arg₁₄₂, Arg₁₆₉, Thr₁₇₀ and Arg₃₇₃ with Val₁₂₈, Arg₁₆₉ and Thr₁₇₀ playing the critical roles in 5′ monophosphate detection. The binding of the RNA substrate by the 5′ sensor is predicted to induce a significant structural conformational change that helps to correctly position the RNA substrate for cleavage.

A truncation of the rne locus, rne131, resulting in an RNase E polypeptide lacking its non-catalytic region (while retaining amino acid residues 1-584) was isolated in a screen for suppressors of a temperature-sensitive allele of the mukB gene (Kido et al 1996). A later study showed that in strains such as BL21(DE3), introducing the rne131 truncation caused a bulk stabilisation of mRNA degradation, including mRNA produced by T7 RNAP (Lopez et al 1999). The rne131 truncation was engineered into the commercially available BL21Star (DE3) with the rationale that stabilising bulk mRNA would result in increased protein production. However, the commercial strain also comes with a note suggesting that it might be unsuitable for the over-expression of toxic genes.

Membrane proteins (MPs) are important drug targets and play essential roles in basic cellular mechanisms. In both prokaryotes and eukaryotes, 20-30% of all genes encode membrane proteins. MPs are involved in fundamental mechanisms such as transport of nutrients and signal molecules, response to environmental changes, membrane stability, maintenance of the redox potential, defence, and energy conversion. With natural abundances often too low to isolate sufficient material for In vitro studies, structural and biochemical investigations are limited by our ability to produce and purify MPs recombinantly in a functional state. MPs are also notoriously known for causing burden in expression systems and are hence considered toxic proteins.

### SUMMARY OF THE INVENTION

In a first aspect, the invention provides a prokaryotic microbial host cell for recombinant expression of a target protein, said cell comprising
a. a gene encoding an enzyme having endoribonuciease activity (E.C. 3.1.26), said enzyme preferably having at least 75% amino acid sequence identity to SEQ ID NO. 2, wherein said gene is on the genome of said cell,
b. a first recombinant gene encoding a mutant RNase E, wherein the amino acid sequence of said mutant RNase E has at least 75% sequence identity with SEQ ID NO. 2, and wherein the amino acid sequence of said mutant RNase E has one or more amino acid residue substitutions which results in the mutant RNAse E having decreased activity compared to the RNase E of SEQ ID NO. 2, and
c. a second recombinant gene encoding said target protein,
wherein expression of said target protein is enhanced compared to a cell lacking said first recombinant gene.

In a second aspect, the invention provides a prokaryotic vector comprising
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions which results in the mutant RNAse E having decreased activity compared to the RNase E of SEQ ID NO. 2, and
b. a gene encoding a T7 lysozyme (E.C. 3.5.1.28)

In a third aspect, the invention provides the use of the prokaryotic vector according to the second aspect of the invention for enhancing expression of a recombinant gene encoding a target protein in a host prokaryotic microbial cell.

In a fourth aspect, the invention provides a method for the production of a target protein comprising culturing in a suitable culture medium, a host cell according to the first aspect of the invention, optionally inducing expression of said target protein, followed by isolation and purification of the expressed target protein by a well-known technique.

In a sixth aspect, the invention provides a method for producing a prokaryotic recombinant microbial cell having enhanced expression of a recombinant target protein,
a. providing a prokaryotic microbial cell comprising (i) a gene encoding an enzyme having endoribonuclease activity (E.C. 3.1.26), said enzyme preferably having at least 75% amino acid sequence identity to SEQ ID NO. 2, wherein said RNase gene is on the genome of said cell, and (II) a gene encoding said recombinant target protein,
b. transforming said microbial cell with a prokaryotic vector comprising a recombinant gene encoding a mutant RNase E having at least 75% amino acid sequence identity to SEQ ID NO. 2, and wherein the amino acid sequence of said mutant RNase E has one or more amino acid residue substitutions which results in the mutant RNAse E having decreased activity compared to the RNase E of SEQ ID NO. 2,
wherein expression of said target protein is enhanced compared to a cell not transformed with said prokaryotic vector.

### DESCRIPTION OF THE INVENTION

### Definitions and Abbreviations

“Amino acid residue substitution” at a specific position means substitution with any amino acid different from the native amino acid residue that is present at that specific position.

Conservative vs nonconservative amino acid substitution: A conservative amino acid substitution replaces an amino acid with another amino acid that is similar in size and chemical properties such that the substitution has no or only minor effect on protein structure and function; meanwhile a nonconservative amino acid substitution replaces an amino acid with another amino acid that is dissimilar and thereby is likely to affect structure and function of the protein.

rne* means a mutant rne gene comprising one or more mutations resulting in one or more amino acid substitutions in the catalytic N-terminal domain of RNase E enzyme and thereby modulates the endoribonuclease activity (E.C. 3.1.26) of the RNase E enzyme, compared to the non-mutated version of the enzyme.

Standard one or three letter abbreviations for amino acids are used herein.

### DESCRIPTION OF FIGURES

FIG. 1A: Illustration of the E. coli RNA degradosome assembled around RNase E. N- and C-terminal domain of the membrane-bound essential endonuclease RNase E ([a] black) and the localisation of associated enzymes Rhlb [b], enolase [c], and PNPase [d] along the C-terminal non-catalytic scaffolding region are displayed. The mutation of the rne gene present on plasmid pMax (rne^{D346N}) as well as the truncation of the rne gene in BL21Star (DE3) are indicated.

FIG. 15: Important elements of the catalytically active N-terminal domain of E. coli RNase E. The location of the different elements along the N-terminal domain (NTD) of E. coli RNase E as well as important amino acid residues forming the uracil pocket, the 5′ sensor pocket, and the catalytic side in the mature protein are indicated.

FIG. 2A: Plasmid illustration. Schematic illustration of plasmids pMax-me^{X}.

FIG. 25: Effect of rne mutations on protein production. Co-expression of me variants on auxiliary plasmid pMax-rne^{X} (illustrated) along with yidC-gfp expression vector pYidC in BL21(DE3). RNase E production is under the control of a rhamnose promotor. YidC-GFP expression is induced via IPTG. The measured fluorescence is proportional to YidC-GFP expression under the different conditions.

FIG. 3A: Plasmid Illustrations. Schematic illustration of plasmids pMax and vectors used for co-expression of GFP fusion proteins: pYidC, p450, pHtpX and pYqiK. Gene names are abbreviated as gene of interest (GOI).

FIG. 3B: Effect of rne mutations on the expression of challenging/toxic proteins. Heterologous production of a variety of “challenging/toxic” GFP-fusion proteins in BL21Star (DE3) harbouring a pLysS plasmid compared to expression of the same genes in BL21(DE3) when co-expressing either the auxiliary plasmid pLysS or pMax. The measured fluorescence is proportional to expression of the GFP-fusion proteins.

FIG. 4: Effect of IysS and rne^{D346N} N co-expression on the expression of challenging/toxic proteins. Heterologous production of “challenging/toxic” GFP-fusion protein YidC-GFP in BL21(DE3) without co-expression of an auxiliary plasmid and when co-expressing either auxiliary plasmid pLysS (lysS), pMax (lysS, rne^{D346N}) or pMax not harbouring a lysS gene (pMax-noLysS: rne^{D345N}, no lysS). The measured fluorescence is proportional to YidC-GFP expression under the different conditions.

FIG. 5: Effect of a truncation of the rne^{D346N} gene present on pMax on protein co-expression. Co-expression of pLysS, pMax (not truncated (“none”)), and C-terminally truncated rne^{D346} variants on pMax (truncated at either position V489 or L529) and expression vector pYidC. The measured fluorescence is proportional to YidC-GFP expression under the different conditions.

FIG. 6: pMax co-expression in BL21Star (DE3). Co-expression of auxiliary plasmid pLysS or pMax with expression vector pYidC in BL21(DE3) and BL21Star (DE3). The measured fluorescence is proportional to YidC-GFP expression under the different conditions.

FIG. 7: Effect of different amino acid substitutions in rne position D346 on the performance of pMax. Co-expression of rne^{D346X} variants on pMax (specific amino acid residue changes are indicated in one-letter-code below each bar), pMax (harbouring rne^{D346N}) or pLysS along with expression vector pYidC. The measured fluorescence is proportional to YidC-GFP expression under the different conditions.

FIG. 8: Effect of different amino acid substitutions across rne on the performance of pMax. Co-expression of rne* variants on pMax (specific amino acid residue changes and positions are indicated in one-letter-code below each bar), pMax (harbouring rne^{D346N}), pLysS-rneWT or pLysS along with expression vector pYidC. The measured fluorescence is proportional to YidC-GFP expression under the different conditions.

FIG. 9: Effect of additional amino acid substitution across rne on the performance of pMax. (A) and (B) Co-expression of rne^{D346N, X} variants on pMax (additional amino acid residue change and position is indicated in one-letter-code below each bar), pMax (harbouring rne^{D345N}), pLysS-rneWT or pLysS along with expression vector pYidC. The measured fluorescence is proportional to YidC-GFP expression under the different conditions.

FIG. 10: Effect of pMax co-expression on Proteinase K production in Shuffle (DE3). Activity of Proteinase K expressed in the cytosol in SHuffle (DE3) co-expressing either plasmid pLysS or pMax. Negative control samples are pure buffer and expression from a pET-empty vector that carries no Proteinase K gene.

FIG. 11: Effect of lysS and rne^{346N} co-expression on the expression of challenging/toxic proteins (YidC) and non-toxic proteins (GFP). Heterologous production of GFP-fusion protein YidC-GFP and GFP in BL21(DE3) when co-expressing either auxiliary plasmid pLysS (lysS) or pMax (lysS, rneD346N). The measured fluorescence is proportional to YidC-GFP expression under the different conditions.

### DETAILED DESCRIPTION OF THE INVENTION

The present invention concerns a method of enhancing recombinant protein expression in microbial organisms by providing a mutant RNase E enzyme on an auxiliary plasmid.

### I. Microbial Host Cell for Recombinant Gene Expression

In one aspect, the present invention provides a microbial host cell for recombinant expression of a target protein.

The microbial host cell of the invention comprises
a. a gene on the genome of said cell encoding an enzyme having endoribonuclease activity (E.C. 3.1.26),
b. a recombinant gene encoding a target protein, and
c. a recombinant gene (rne*) encoding a mutant RNase E enzyme, and
wherein expression of said target protein is enhanced compared to a cell lacking said recombinant gene (rne*) encoding said mutant RNase E enzyme.

RNase activity is generally considered an important activity of a microbial cell. In some microorganisms (e.g. E. coli), knocking out RNase genes leads to non-viable cells. RNase activity in a host cell affects recombinant protein expression. The present invention provides a means for regulating RNase activity by providing an RNase E mutant on an auxiliary plasmid. The activity of the RNase E mutant is modulated through one or more point mutations in the N-terminal catalytic region of the RNase E enzyme. Without wishing to be bound by theory, it is believed that the mutant RNase E enzyme thereby acts as a competitive inhibitor of the native RNases, as the mutant RNase E is still able to engage with RNA, leading to less degradation of RNA due to its decreased activity. As disclosed in the background section, the active RNase E degradosome is a tetramer of the individually expressed RNAse E enzymes; the homotetramer primarily being formed by interactions between the small domains of the RNAse Es. In the microbial host cell of the invention, the homotetramer may comprise 1, 2, 3, or 4 mutant RNase E enzymes (the remaining units being native RNase E enzyme). Without wishing to be bound by theory, further to the above, it is believed that the one or more mutant RNase E enzyme in the homotetramer degradosome are dominant over the native RNase E enzyme and thereby decrease the overall activity of the degradosome complex. Expression levels of the mutant RNase E can be regulated by selecting suitable promoters and RBS sequences, and can thereby be tuned to outcompete native RNases.

### I.i Host Cell

In one embodiment, the host cell is a prokaryotic microbial host cell, such as Escherichia coil, Pseudomonas putida, Bacillus subtilis and Bacillus licheniformis. In one embodiment, the prokaryotic microbial host cell is selected from gram-negative bacteria, such as Escherichia coli and Pseudomonas putida. In one embodiment, the prokaryotic host cell is selected from gram-positive bacteria, such as Bacillus subtilis. In a preferred embodiment, the prokaryotic microbial host cell is selected from E. coli and B. subtills. In a further preferred embodiment, the host cell is selected from E. coli strain BL21, BL21(DE3), and BL21Star (DE3), or K-12 MG1655; preferably strain BL21(DE3).

In another embodiment, the host cell is a eukaryotic microbial host cell. In one embodiment, the eukaryotic microbial host cell is selected from Saccharomyces cerevisiae, Aspergillus niger, and Pichia pastoris.

In yet another embodiment, the host cell is a mammalian cell, in one embodiment, the mammalian cell is selected from CHO and HEK cell lines.

### I.ii Genomic Gene Encoding Endoribonuclease Activity

In one embodiment, the microbial host cell of the invention comprises a gene on its genome encoding an enzyme having endoribonuclease activity (E.C. 3.1.26).

In one embodiment, the prokaryotic microbial host cell of the invention comprises a gene encoding an enzyme having endoribonuclease activity (E.C. 3.1.26) on its genome. In one embodiment, said enzyme having endoribonuclease activity (E.C. 3.1.26) has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8.

In one embodiment, the prokaryotic microbial host cell of the invention comprises a gene encoding an RNase E enzyme (E.C. 3.1.26.12) on its genome. In one embodiment, the RNase E enzyme encoded by a gene on the genome of the host cell has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2 or 4.

In general, the sequence of the catalytic region of RNase E is highly conserved among Gram-negative bacteria, but some properties of the catalytic domain may be species-specific. For gram-negative bacteria, in one embodiment, the RNase E enzyme encoded by a gene on the genome of the host cell has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% amino acid sequence identity to a RNase E native to said prokaryotic host cell.

For example, where the prokaryotic host cell is E. coli, the host cell comprises a gene on its genome encoding a RNase E (E.C. 3.1.26.12) having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2. For example, where the prokaryotic host cell is Pseudomonas putida, the host cell comprises a gene on its genome encoding a RNase E (E.C. 3.1.26.12) having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 4.

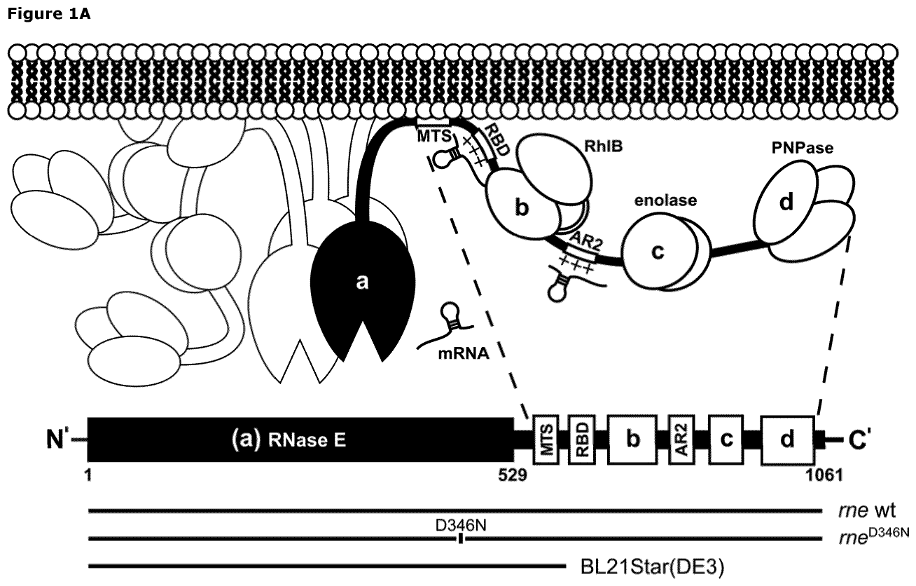

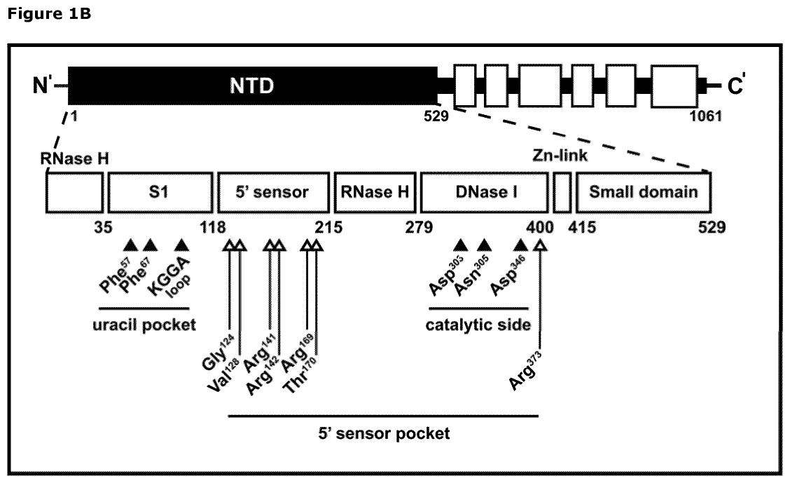

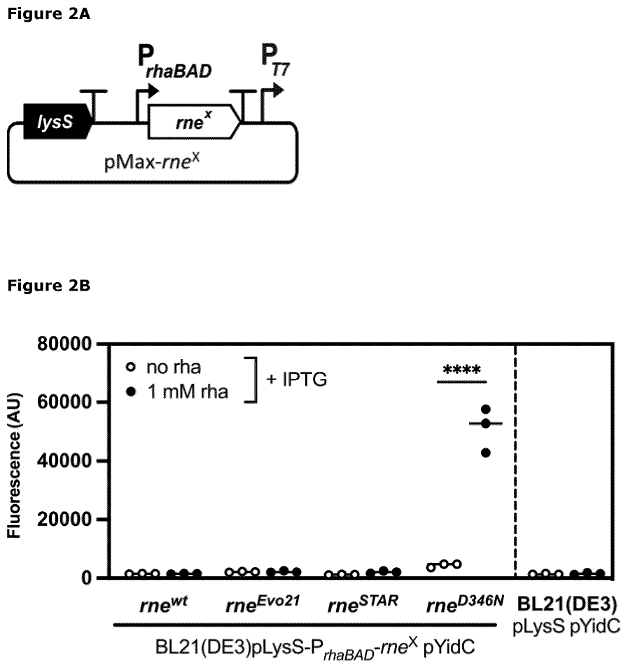

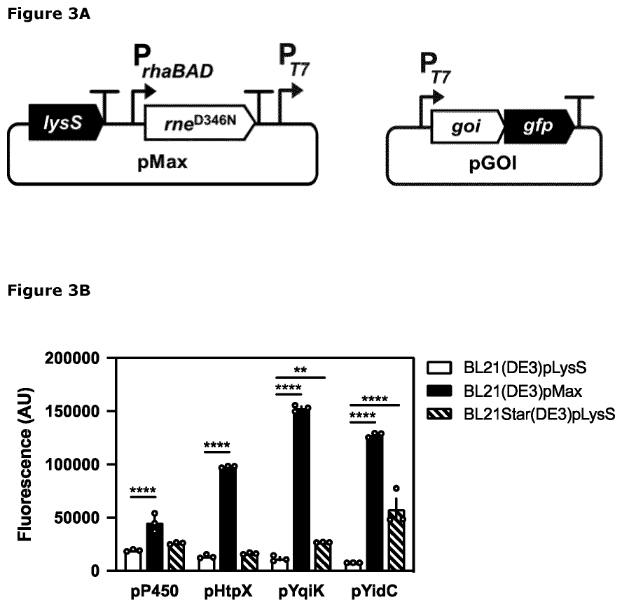

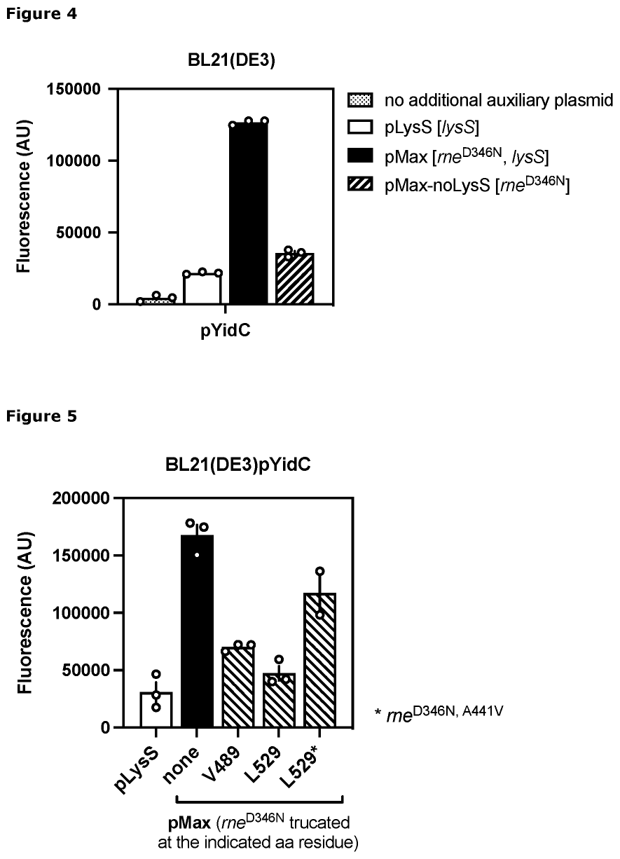

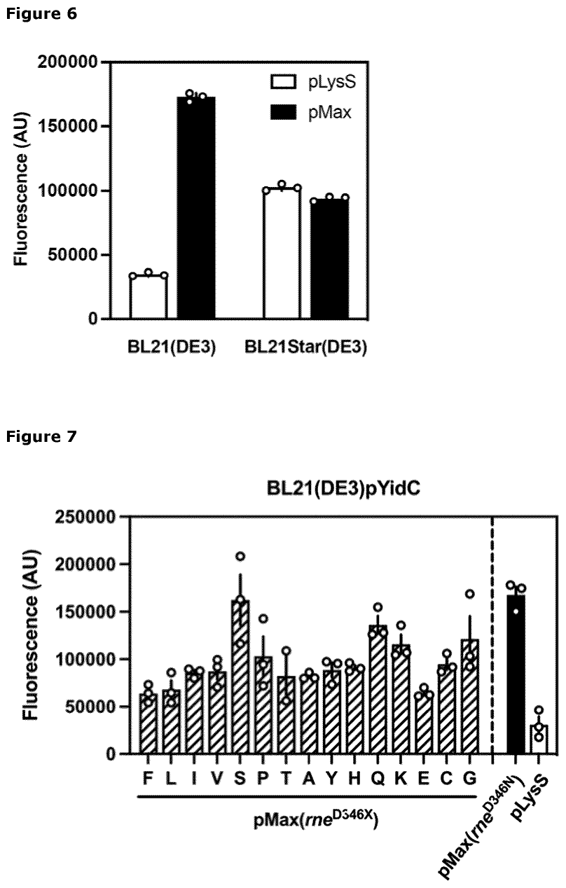

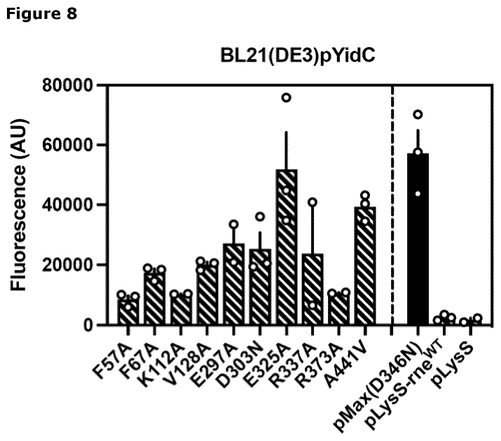

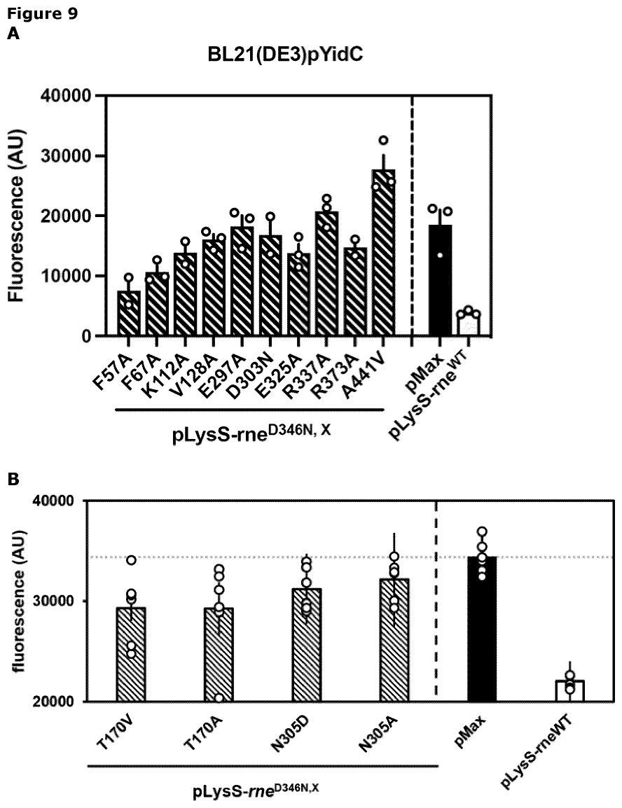

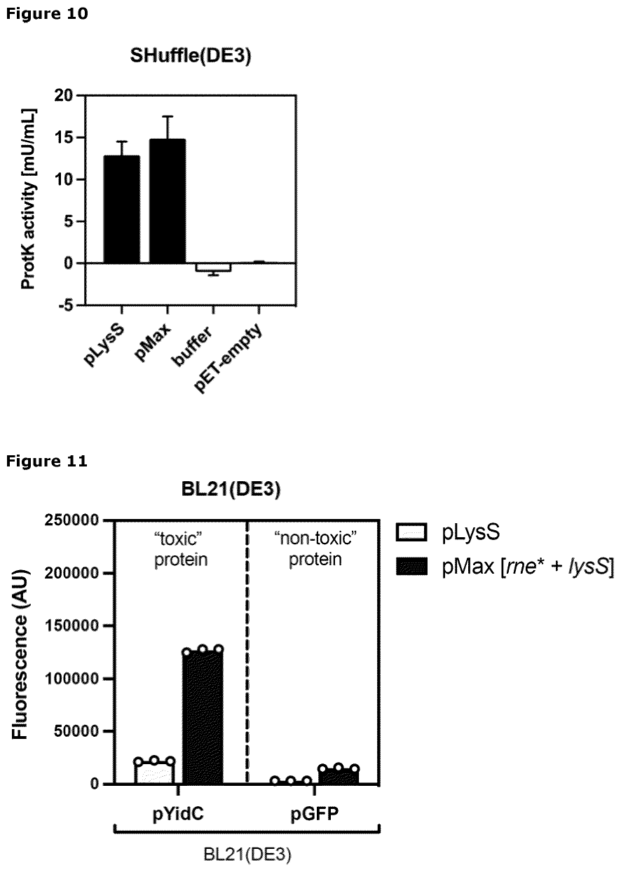

Preferably, the RNase E gene on the genome of the microbial host cell is full length, compared to the native RNase E of said host cell, i.e not truncated, and thereby comprises both its N-terminal catalytic domain and its C-terminal non-catalytic domain. For illustrations of E. coli rne domains, see FIG. 1.

Gram-positive bacteria do not possess any native homologs of RNase E. For example in B. subtills, RNA degradation is done via RNases J1/J2 and Y which form a complex with additional enzymes similar to the degradosome in E. coli mediated via RNase E. Whereas RNase J2 seems to possess a similar function and structure as RNase E but no homology, B. subtilis RNase Y can be functionally replaced by E. coli RNase E. Full-length RNase E almost completely restores wild type growth of an rny null mutant. RNase E (E. coli) and RNase Y (B. subtilis) require a Mg²⁺ ion to function and are involved in the initiation of mRNA decay. Although the amino acid sequence of RNase Y shows a low identity to that of RNase E, they share the same function as endo-ribonucleases with relaxed sequence specificity. Additionally, a degradosome-like complex centred around RNase Y has been proposed.

For gram-positive bacteria, in one embodiment, the genome of the host cell comprises a gene encoding an enzyme having endo-ribonuclease activity (E.C. 3.1.26). In one embodiment, such enzyme has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% amino acid sequence identity to a RNase Y enzyme native to said gram-negative host cell. For example, where the prokaryotic host cell is Bacillus subtilis, the host cell comprises a gene on its genome encoding a RNase Y having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 6. For example, where the prokaryotic host cell is Bacillus licheniformis, the host cell comprises a gene on its genome encoding a RNase Y having at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 8. Fungal RNases such as RNases of Saccharomyces, Pichia, and Aspergilli species mostly belong to the RNase T1 family, that do not share sequence identity as such with the E. coli RNase E. Major RNases important in S. cerevisiae, Pichia, and Aspergilli include major cytoplasmic deadenylase CCR4 (gene expression regulation and poly-A shortening/mRNA decay), Pan2/Pan3 complex (mRNA deadenylase), and especially Rpb4/Rpb7 (cytosolic mRNA decay).

In one embodiment, the eukaryotic microbial host cell of the invention comprises one or more gene(s) encoding RNase(s) belonging to the RNase T1 family on its genome. In one embodiment, the one or more RNases encoded on the genome of the eukaryotic host cell has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% amino acid sequence identity to a RNase native to said eukaryotic host cell.

For example, where the eukaryotic host cell is Saccharomyces cerevisiae, the host cell comprises a gene encoding major cytoplasmic deadenylase CCR4, Pan2/Pan3 complex and/or Rpb4/Rpb7 on its genome on its genome. In one embodiment, said CCR4 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 10, said Pan2 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 12, said Pan3 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 14, said Rpb4 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 16, and said Rpb7 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 18,

For example, where the eukaryotic host cell is Pichia pastoris, the host cell comprises a gene encoding major cytoplasmic deadenylase CCR4, Pan2/Pan3 complex and/or Rpb4/Rpb7 on its genome on its genome. In one embodiment, said CCR4 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 19, said Pan2 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 20, said Pan3 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 21, said Rpb4 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 22, and said Rpb7 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 23,

For example, where the eukaryotic host cell is Aspergillus niger, the host cell comprises a gene encoding major cytoplasmic deadenylase CCR4, Pan2/Pan3 complex and/or Rpb4/Rpb7 on its genome on its genome. In one embodiment, said CCR4 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 24, said Pan2 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 25, said Pan3 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 26, said Rpb4 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 27, and said Rpb7 has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 28,

### I.iii Recombinant Gene Encoding Target Protein

The microbial cell of the invention comprises one or more recombinant gene(s) encoding one or more target protein(s). The term protein may refer to any peptide, polypeptide or protein.

The recombinant gene may be provided on a plasmid or incorporated into the genome of the microbial host cell. Such plasmid comprising the recombinant gene encoding the target protein shall further comprise commonly known functionalities for maintenance of the plasmid in the cell, such as an origin of replication—as recognized by a person skilled in the art. The number of copies of the recombinant gene in the microbial cell may be regulated by placing the gene on a plasmid with a high or low copy number.

The recombinant gene encoding the target protein may be inducible or constitutively expressed. In a preferred embodiment, expression of the recombinant gene is regulated by an inducible promoter. Any inducible promoter may be used. The induction of protein expression in bacteria is well known in the art. In one embodiment of the present invention, the induction of protein expression is for example made by the addition of isopropyl-β-D-thioyalactopyranoside (IPTG), as specified below.

In one embodiment, the microbial host cell comprises a recombinant gene encoding a target protein and a gene encoding a T7 RNA polymerase (E.C. 2.7.7.6), wherein expression of said recombinant gene is regulated by an inducible T7 promoter.

In expression of the recombinant gene encoding the target protein regulated by such T7 system, the T7 RNA polymerase (RNAP) may be under the control of an inducible promoter, such as a lac promoter inducible by IPTG. Further, the T7 promoter regulating the expression of the recombinant gene may be regulated by the function of a lac operon (hence inducible by IPTG), for example by means of a lac repressor and lac operator.

In a further embodiment, the microbial host cell further comprises a gene encoding a T7 lysozyme (E.C. 3.5.1.28). T7 lysozyme is a natural inhibitor of the T7 RNA polymerase. In one preferred embodiment, the microbial host cell comprises a gene encoding a T7 RNA polymerase (E.C. 2.7.7.6), a gene encoding a T7 lysozyme (E.C. 3.5.1.28), and a recombinant gene encoding a target protein, wherein expression of said recombinant gene is regulated by an inducible T7 promoter.

The T7 lysozyme is especially useful in systems, where inducible expression of the T7 RNA polymerase is “leaky”. The “DE3” strains of E. coli comprise such leaky T7 RNA polymerase expression, as it is regulated by the IPTG inducible lacUV5 promoter, but which is known to be leaky and thereby allow for some basal expression of the T7 RNA polymerase.

In one embodiment, the microbial host cell is an E. coli strain comprising the T7 system, such as the “DE3” strains, where the recombinant gene encoding the target protein is provided on a pET vector, such as pET28a+ (SEQ ID NO. 46), preferably in combination with expression of a gene encoding T7 lysozyme (E.C. 3.5.1.28), such as LysS (SEQ ID NO. 36) provided on a second plasmid—e.g. pMax (SEQ ID NO. 38) of the present invention, which comprises pLysS SEQ ID No. 29) as the backbone.

In yet another embodiment, expression of the recombinant gene encoding the target protein is regulated by a promoter which is recognized by the host's native polymerase, this promoter is preferably an inducible promoter.

In one embodiment, the promoter for regulating the expression of the recombinant gene encoding the target protein is selected from rhaBAD promoter (SEQ ID NO 41), araBAD promoter (SEQ ID NO 88), Ptrc promotor (SEQ ID NO 89), Ptet promoter (SEQ ID NO 90), Ptac promoter (SEQ ID NO 91), and PL promoter (SEQ ID NO 92).

In one embodiment, said promoter is native to the host cell.

### I.iv Recombinant Gene (Rne*) Encoding a Mutant RNase E

The microbial host cell of the invention comprises a recombinant gene rne* encoding a mutant RNase E enzyme.

In one embodiment, the microbial cell comprises a mutant RNase E enzyme having one or more amino acid residue substitutions, which facilitates improved expression of a target protein, preferably a toxic protein such as YidC (SEQ ID NO. 68), compared to expression of said target protein in a parent cell (from which the microbial cell was derived) lacking expression of the mutant RNase E.

In one preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, wherein the amino acid sequence of said mutant RNase E has one or more amino acid residue substitutions, preferably a dominant substitution, wherein said one or more amino acid residue substitutions facilitates improved expression of a target protein, preferably a toxic protein such as YidC (SEQ ID NO. 68), compared to expression of said target protein in a parent cell (from which the microbial cell was derived) lacking expression of the mutant RNase E.

A person skilled in the art will recognize that such amino acid residue substitution which facilitates improved expression of a target protein may be identified by expressing a candidate mutant RNAse E comprising a candidate amino acid substitution in the microbial cell together with the target protein, preferably a toxic protein such as YidC (SEQ ID NO. 68), and confirming improved expression of YidC compared to a parent cell (from which the microbial cell was derived) lacking expression of the candidate mutant RNase E.

In one embodiment, the microbial host cell of the invention comprises a vector comprising a recombinant gene rne* encoding a mutant RNase E enzyme. The vector may be a prokaryotic or eukaryotic plasmid, comprising an origin of replication suitable for replication in the respective prokaryotic or eukaryotic host cell, independently of the chromosome.

In one embodiment, the microbial host cell of the invention comprises a recombinant gene rne* encoding a mutant RNase E enzyme on its genome.

As mentioned previously, an essential function of RNase E is mRNA degradation; the N-terminal domain (NTD) of RNase E is responsible for this endoribonuclease activity. The rne* gene comprises one or more point mutations resulting in one or more amino acid substitutions in the catalytic N-terminal domain of the RNase E enzyme, thereby modulating the activity of the RNase E enzyme, compared to the non-mutated version of the enzyme.

As mentioned previously, the catalytic N-terminal domain of RNase E is highly conserved. In one embodiment, the recombinant rne* gene encoding mutant RNase E comprises an amino acid substitution in the DNAse I domain. In one embodiment, the amino acid substitution affects metal ion chelation in the RNase E enzyme.

Table 1 provides a list of residues of E. coli and P. putida RNase E relevant for the present invention and describes the function in E. coli RNase E when mutated. In a preferred embodiment, the mutant RNase E encoded by rne* comprises one or more amino acid substitution(s) at one or more positions relative to the positions listed in table 1. A person skilled in the art would know how to perform sequence alignment of homologous RNase E sequences from different organisms to identify these specific positions with reference to the information provided in table 1.

TABLE 1
Relevant residues of E. coli RNase E (SEQ
ID NO. 2) and P. putida (SEQ ID NO. 4)

| Position | Position | Location | Predicted function of mutant | |
|---|---|---|---|---|
| in E. coli | in P. putida | (FIG. 1B) | forms of E. coli RNase E | Ref. |
| D346 | D343 | DNase I | Metal ion chelation - activity | 1 |
| | | | decrease | |
| E297 | E294 | DNase I | Metal ion chelation (similar to | 1 |
| | | | D346N) - active | |
| D303 | D300 | DNase I | Metal ion chelation (same as for | 1 |
| | | | D346N); activity decrease, nearly | |
| | | | inactive | |
| N305 | N302 | DNase I | Activity decrease (supports D303 | 1, 2 |
| | | | through hydrogen bonding) | |
| E325 | E322 | DNase I | Metal ion chelation (similar to | 1 |
| | | | D346N) - active | |
| R337 | R334 | DNase I | Metal ion chelation (similar to | 1 |
| | | | D346N); highly conserved | |
| D349 | D346 | DNase I | Metal ion chelation (similar to | 1 |
| | | | D346N) - active | |
| V128 | V126 | 5′ sensor | Removes enhancement of cleavage | 2 |
| | | | seen for substrate with 5′ | |
| | | | monophosphate; highly conserved; | |
| | | | inactive | |
| R169 | R167 | 5′ sensor | Removes enhancement of cleavage | 1, 2 |
| | | | seen for substrate with 5′ | |
| | | | monophosphate | |
| T170 | T168 | 5′ sensor | Inactive | 2 |
| F57 | F57 | S1 | 50-fold activity decrease | 2 |
| F67 | F67 | S1 | 50-fold activity decrease | 2 |
| K112 | K110 | S1 | 50-fold activity decrease | 2 |
| G124 | G122 | 5′ sensor | 5′ sensor/RNA binding, possibly | 4 |
| | | | decreased function or inactive | |
| R141 | R139 | 5′ sensor | 5′ sensor/RNA binding, possibly | 4 |
| | | | decreased function or inactive | |
| R142 | R140 | 5′ sensor | 5′ sensor/RNA binding, possibly | 4 |
| | | | decreased function or inactive | |
| R373 | R370 | DNase I | 5′ sensor/RNA binding, possibly | 4 |
| | | | decreased function or inactive | |
| A441 | A438 | Small domain | unknown | This |
| | | (C-terminal) | | study |

Ref:
1 Garey et al 2009,
2 Callaghan et al 2005,
³Kim et al 2014,
4 Mardle et al 2019.

Mutations at residues D346, E297, D303, N305, E325, R337, D349, V128, R169, T170, F57, F67, K112, G124, R141, R142, or R373 are based on prior art predicted to decrease the activity of RNase E.

Residues F57, F67, K112, D303, and D346 in E. coli RNase E are active site residues (Garey et al 2009).

Residues D346, E297, D303, N305, E325, R337, and D349 are important for metal ion chelation. Specifically residues D346, E297, D303, E325, R337, and D349 within the DNAse I domain are important for metal ion chelation (Garey et al 2009), while residue N305 supports D303 through hydrogen bonding (Callaghan et al 2005).

Residues G124, V128, R141, R142, R169, T170, R373, F57, F67, K112 are important for RNA recognition (5′ sensor domain and other domains). Several residues in the S1 domain could contribute to RNA binding, but only three residues: F57, F67, and K112 provide obvious contacts to the substrate (Garey et al 2009). Therefore, specifically residues F57, F67, K112 are important, as they are in contact with RNA (S1 domain). Residues E297, D303, N305, E325, R337, D346, D349, R373 are important DNase I-like domain residues.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has one or more amino acid residue substitutions which results in the mutant RNAse E having decreased activity compared to the RNase E of SEQ ID NO. 2.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, wherein the amino acid sequence of said mutant RNase E has one or more amino acid residue substitutions which results in the mutant RNAse E having decreased activity compared to the RNase E of SEQ ID NO. 2, and wherein said one or more amino acid residue substitutions facilitates improved expression of a target protein, preferably a toxic protein such as YidC (SEQ ID NO. 68), compared to expression of said target protein in a parent cell (from which the microbial cell was derived) lacking expression of the mutant RNase E.

In one embodiment, the activity of the mutant RNAse E is decreased by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% compared to the RNase E of SEQ ID NO. 2 when expressed in a host cell of the invention. In one embodiment, the activity of the mutant RNAse E is decreased at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold compared to the RNase E of SEQ ID NO. 2 when expressed in a host cell of the invention.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has one or more amino acid residue substitutions at one or more positions selected from D346, E297, D303, N305, E325, R337, D349, V128, R169, T170, F57, F67, K112, G124, R141, R142, R373, and A441 relative to SEQ ID NO. 2

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution which reduces the metal ion chelation ability of the enzyme. In a preferred embodiment, said metal ion chelation ability is reduced by substituting an amino acid at a position selected from D346, E297, D303, N305, E325, R337, and D349 relative to SEQ ID NO. 2.

In a preferred embodiment, the amino acid residue substitutions mentioned above are non-conservative substitutions.

In one preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution which results in the mutant RNAse E having reduced metal ion chelation ability compared to the RNase E of SEQ ID NO. 2. In a preferred embodiment, said metal ion chelation ability is reduced by substituting an amino acid at a position selected from D346, E297, D303, E325, R337, and D349 relative to SEQ ID NO. 2.

In one embodiment, the metal ion chelation ability of the mutant RNAse E is decreased by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% compared to the RNase E of SEQ ID NO. 2 when expressed in a host cell of the invention. In one embodiment, the metal ion chelation ability of the mutant RNAse E is decreased at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold compared to the RNase E of SEQ ID NO. 2 when expressed in a host cell of the invention.

In a preferred embodiment, the microbial cell comprising a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has a single amino acid residue substitution at position D346 relative to SEQ ID NO. 2, wherein said amino acid is substituted to any amino acid other than aspartate. The essential aspartate residue in position 346 in the so-called DNase I subdomain of the native RNase E is involved in chelating an essential Mg⁺² ion. The aspartate residue is predicted to act as a general base to activate the attacking water essential for the catalytic activity of the enzyme (Callaghan et al 2005). The replacement of Asp-346 with the polar amino acid Asn was previously shown to decrease RNA cleavage by about 25-fold (Callaghan et al 2005).

In yet a preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has a single amino acid residue substitution at position D346 relative to SEQ ID NO. 2, wherein said amino acid is substituted to asparagine.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution which reduces the RNA recognition ability of the enzyme. In one such embodiment, said RNA recognition ability is reduced by substituting an amino acid at a position selected from G124, V128, R141, R142, R169, T170, R373, F57, F67, and K112 relative to SEQ ID NO. 2.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution which modifies an RNA contact point of the enzyme. In one such embodiment, said RNA contact point is modified by substituting an amino acid at a position selected from F57, F67, and K112 relative to SEQ ID NO. 2.

In one preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution which results in the mutant RNAse E having a modified RNA contact point compared to the RNase E of SEQ ID NO. 2. In a preferred embodiment, said substituting for modifying the RNA contact point is an amino acid at a position selected from F57, F67, and K112relative to SEQ ID NO. 2.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution in the 5′ sensor pocket of the enzyme, preferably the pocket ‘anchors’. In one such embodiment, said pocket ‘anchor’ is modified by substituting an amino acid at a position selected from V128, and R373 relative to SEQ ID NO. 2.

In yet a preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 2, and wherein the amino acid sequence of said mutant RNase E has an amino acid residue substitution at position A441 relative to SEQ ID NO. 2.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has one or more amino acid residue substitutions which results in the mutant RNAse E having decreased activity compared to the RNase E of SEQ ID NO. 4.

In one embodiment, the activity of the mutant RNAse E is decreased by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% compared to the RNase E of SEQ ID NO. 4 when expressed in a host cell of the invention. In one embodiment, the activity of the mutant RNAse E is decreased at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold compared to the RNase E of SEQ ID NO. 4 when expressed in a host cell of the invention.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has one or more amino acid residue substitutions at one or more positions selected from D343, E294, D300, N302, E322, R334, D346, V126, R167, T168, F57, F67, K110, G122, R139, R140, R370, and A438 relative to SEQ ID NO. 4.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution which reduces the metal ion chelation ability of the enzyme. In a preferred embodiment, said metal ion chelation ability is reduced by substituting an amino acid at a position selected from D343, E294, D300, N302, E322, R334, and D346 relative to SEQ ID NO. 4.

In one preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution which results in the mutant RNAse E having reduced metal ion chelation ability compared to the RNase E of SEQ ID NO. 4. In a preferred embodiment, said metal ion chelation ability is reduced by substituting an amino acid at a position selected from D343, E294, D300, E322, R334, and D346 relative to SEQ ID NO. 4.

In one embodiment, the metal ion chelation ability of the mutant RNAse E is decreased by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% compared to the RNase E of SEQ ID NO. 4 when expressed in a host cell of the invention. In one embodiment, the metal ion chelation ability of the mutant RNAse E is decreased at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold compared to the RNase E of SEQ ID NO. 4 when expressed in a host cell of the invention.

In a preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has a single amino acid residue substitution at position D343 relative to SEQ ID NO. 4, wherein said amino acid is substituted to any amino acid other than aspartate.

In yet a preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has a single amino acid residue substitution at position D343 relative to SEQ ID NO. 4, wherein said amino acid is substituted to asparagine.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution which reduces the RNA recognition ability of the enzyme. In one such embodiment, said RNA recognition ability is reduced by substituting an amino acid at a position selected from G122, V126, R139, R140, R167, T168, R370, F57, F67, and K110 relative to SEQ ID NO. 4.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution which modifies an RNA contact point of the enzyme. In one such embodiment, said RNA contact point is modified by substituting an amino acid at a position selected from F57, F67, and K110 relative to SEQ ID NO. 4.

In one preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution which results in the mutant RNAse E having a modified RNA contact point compared to the RNase E of SEQ ID NO. 2. In a preferred embodiment, said substituting for modifying the RNA contact point is an amino acid at a position selected from F57, F67, and K110 relative to SEQ ID NO. 4.

In one embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution in the 5′ sensor pocket of the enzyme, preferably the pocket ‘anchors’. In one such embodiment, said pocket ‘anchor’ is modified by substituting an amino acid at a position selected from V126, and R370 relative to SEQ ID NO. 4.

In yet a preferred embodiment, the microbial cell comprises a recombinant gene encoding a mutant RNase E, wherein the amino acid sequence of said mutant RNase E has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 4, and wherein the amino acid sequence of said mutant RNase E has an amino acid residue substitution at position A438 relative to SEQ ID NO. 4.

As mentioned previously, though not being homologous, B. subtilis RNase Y can be functionally replaced by E. coli RNase E.

In one embodiment, the microbial cell comprises a recombinant gene encoding a mutant RNase Y, wherein the amino acid sequence of said mutant RNase Y has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 6, and wherein the amino acid sequence of said mutant RNase Y has one or more amino acid residue substitutions which results in the mutant RNAse Y having decreased activity compared to the RNase Y of SEQ ID NO. 6.

In one embodiment, the activity of the mutant RNAse Y is decreased by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% compared to the RNase E of SEQ ID NO. 6 when expressed in a host cell of the invention. In one embodiment, the activity of the mutant RNAse Y is decreased at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold compared to the RNase E of SEQ ID NO. 6 when expressed in a host cell of the invention.

No amino acid positions for B. subtilis RNase Y can be found which are homologue to amino acid positions for E. coli RNase E listed in table 1, however, the His-Asp doublet conserved in HD domain proteins (such as RNase Y) in amino acid position H368 and D369 of B. subtilis RNase Y is similarly involved in metal chelation as D346 in E. coli RNase E. RNase Y mutants comprising amino acid substitutions H368A or D369A show lower activity/cleavage than RNase Y wild type (Shahbabian et al 2009).

In one preferred embodiment, the microbial cell comprises a recombinant gene encoding a mutant RNase E, wherein the amino acid sequence of said mutant RNase Y has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 6, and wherein the amino acid sequence of said mutant RNase Y has an amino acid residue substitution which results in the mutant RNAse Y having reduced metal ion chelation ability compared to the RNase Y of SEQ ID NO. 6.

In one embodiment, the metal ion chelation ability of the mutant RNAse Y is reduced by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% compared to the RNase E of SEQ ID NO. 6 when expressed in a host cell of the invention. In one embodiment, the metal ion chelation ability of the mutant RNAse Y is reduced at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold compared to the RNase E of SEQ ID NO. 6 when expressed in a host cell of the invention.

In one embodiment, the microbial cell comprises a recombinant gene encoding a mutant RNase Y, wherein the amino acid sequence of said mutant RNase Y has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 6 and wherein the amino acid sequence of said mutant RNase Y has an amino acid residue substitution at a position selected from H368 and D369 relative to SEQ ID NO. 6.

In one embodiment, the microbial cell comprises a recombinant gene encoding a mutant RNase Y, wherein the amino acid sequence of said mutant RNase Y has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO. 6, and wherein the amino acid sequence of said mutant RNase Y has an amino acid residue substitution selected from H368A and D369A relative to SEQ ID NO. 6.

In one embodiment, the decreased activity, reduced metal ion chelation ability, modified RNA contact point, etc. for the mutant RNase E (or RNase Y) described above is relative to an RNase E (or RNase Y) having SEQ ID NOs referred to herein. In another embodiment, the decreased activity, reduced metal ion chelation ability, modified RNA contact point, etc. for the mutant RNase E (or RNase Y) described above is relative to the same identical RNase E (or RNase Y) enzyme, but lacking the specific mutation. In yet another embodiment, the decreased activity, reduced metal ion chelation ability, modified RNA contact point, etc. for the mutant RNase E (or RNase Y) described above is relative to the activity of the endoribonuclease enzyme encoded on the genome of the microbial host cell.

In a further embodiment, in addition to the rne* gene comprising one or more point mutations as disclosed herein resulting in one or more amino acid substitutions in the catalytic N-terminal domain of RNase E enzyme modulating the activity of the RNase E enzyme compared to the non-mutated version of the enzyme, said host cel further comprises a gene encoding T7 lysozyme (LysS) (E.C. 3.5.1.28). In one embodiment, plasmid pLyS (SEQ ID NO. 29, see table 3 for further specifications) serves as the backbone for a vector comprising the recombinant rne* gene.

Expression of said mutant RNase E may be constitutive or regulated by an inducible promoter, such as the rhaBAD promoter (SEQ ID NO 41), araBAD promoter (SEQ ID NO 88), Ptrc promotor (SEQ ID NO 89), T7 promoter, (SEQ ID NO 47), Ptet promoter (SEQ ID NO 90), Ptac promoter (SEQ ID NO 91), PL promoter (SEQ ID NO 92).

Expression of said mutant RNase E may further be regulated by optimizing the translational strength of the rne* gene. In bacteria, translational strength is defined by the Shine-Dalgarno/ribosome binding site (RBS) sequence directly upstream of the start codon, while in eukaryotic cells translation initiation regions/Kozak elements can be used to modify translational strength. RBSs in E. coli conferring a broad range of translational strengths can be found in the literature, e.g. Bonde et al, 2016. A skilled person in the art can optimize the translational strength of the gene by constructing variants of the ribosomal binding site and testing which variant performs better.

In a preferred embodiment, the host cell of the invention comprises plasmid pMax (SEQ ID NO. 38) comprising the recombinant rne* gene encoding a mutant RNase E.

### II. Auxiliary Plasmid Comprising Recombinant Gene Encoding Mutant RNase E

In one aspect, the invention provides a plasmid comprising a recombinant rne* gene encoding a mutant RNase E and a lysS gene encoding T7 lysozyme (E.C. 3.5.1.28), as described herein.

Any of the above mentioned favorable rne mutations may be exploited on a plasmid comprising (a) a gene encoding the mutant rne enzyme as well as (b) a gene encoding a T7 lysozyme.

The plasmid may be prokaryotic or eukaryotic, comprising an origin of replication suitable for replication in the respective prokaryotic or eukaryotic host cell, independently of the chromosome.

In one embodiment, the plasmid comprises
a. a gene encoding a mutant RNase E having at least 75% amino acid sequence identity to SEQ ID NO. 2, wherein the amino acid sequence of said mutant RNase E has one or more amino acid residue substitutions which results in the mutant RNAse E having decreased activity compared to the RNase E of SEQ ID NO. 2, and
b. a gene encoding a T7 lysozyme (E.C. 3.5.1.28)

In a preferred embodiment, the plasmid comprises
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions at one or more positions selected from D346, E297, D303, N305, E325, R337, D349, V128, R169, T170, F57, F67, K112, G124, R141, R142, R373, and A441 relative to SEQ ID NO. 2, and
b. a gene encoding a T7 lysozyme (E.C. 3.5.1.28)

In a further preferred embodiment, the plasmid the amino acid residue at position 346 of said mutant RNase E relative to SEQ ID NO. 2 is asparagine.

In one embodiment, the plasmid comprises
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions at one or more positions in the DNAse I-like domain, and
b. a gene encoding a T7 lysozyme (E.C. 3.5.1.28)

In one embodiment, the plasmid comprises
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions which results in a mutant RNAse E having reduced metal ion chelation ability, and
b. a gene encoding a T7 lysozyme (E.C. 3.5.1.28)

In one embodiment, the plasmid comprises
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions which results in a mutant RNAse E having a modified RNA contact point, and
b. a gene encoding a T7 lysozyme (E.C. 3.5.1.28)

In one embodiment, the plasmid comprises
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions at one or more positions in the 5′ sensor pocket, preferably the pocket ‘anchors’, and
b. a gene encoding a T7 lysozyme (E.C. 3.5.1.28)

In one embodiment, plasmid pLyS (SEQ ID NO. 29) serves as the backbone for the plasmid comprising the recombinant rne* gene. In one preferred embodiment, the plasmid comprising a recombinant rne* gene encoding a mutant RNase E of the invention is pMax (SEQ ID NO. 38).

Preparation of plasmid comprising the mutant rne gene may be done by any suitable cloning technique known by a person skilled in the art.

### III Target Protein(s) of the Invention

The microbial host cell of the present invention expressing an auxiliary plasmid comprising mutant rne* as described herein has the advantage of providing a more robust and high yielding process for the production of target proteins, compared to industrially common used strains derived from E. coli BL21(DE3), e.g. BL21(DE)pLysS or BL21Star (DE3).

The present invention is particularly suitable for the expression of proteins that cause a burden and negatively affect the fitness of the cell in other commonly used expression systems. A commonly used term for such proteins is ‘toxic proteins’. Some proteins may be difficult to express due to their size, complexity in folding, aggregation issue, etc. or their expression may cause problems with resource competition for other essential proteins or genes in the cell.

A person skilled in the art will recognize that the burden refers to product-specific metabolic toxicity which a microbial host cell genetically engineered to synthesize the target protein experiences during production of the product under production conditions, and which results in a fitness cost that can be quantified by measuring the percent reduction in the maximum exponential growth rate of the cell (along the growth curve) during production of the product under production conditions as compared to a parent microbial cell devoid or incapable of said production when grown under comparable production conditions.

Expression of the target protein is significantly enhanced when expressed in a host cell of the invention, compared to expression of said protein in another host cell lacking the rne* gene as disclosed herein.

In one embodiment, the target protein of the invention is a protein the expression of which is enhanced by at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100% when expressed in a host cell of the invention, compared to expression of said target protein in the same host cell lacking the rne* gene or the rne* in combination with LysS, as disclosed herein.

The target protein of the invention is a protein the expression of which is enhanced at least 1, 2, 3, 4, 5, 6, 7, 8, 9, or 10 fold, such as even 20, 40, 60, 80, 100 fold, such as even 200, 300, 400, 500, 600, 700, 800, 900 or 1000 fold when expressed in a host cell of the invention, compared to expression of said target protein in the same host cell lacking the rne* gene or the rne* in combination with LysS, as disclosed herein.

In one embodiment, the target protein is selected from membrane proteins, antibody-like proteins, industrial enzymes such as carbohydrases, proteases, and lipases; and pharmaceutical proteins such as peptides, hormones, and proteins for vaccine development. The present invention is widely applicable of a range of different proteins which have proven difficult to express using other common expression systems. As mentioned in the background section, expression of membrane proteins is considered ‘toxic’ for the host cell, and they are notoriously known for causing burden in expression systems. Hence, in a preferred embodiment, the target protein is a membrane protein.

In one embodiment, the target protein is a soluble protein; in another embodiment the target protein is an insoluble protein. In one embodiment the target protein is a secreted protein; in another embodiment the target protein is a non-secreted protein. In one embodiment the target protein is of eukaryotic origin; in another embodiment the target protein is of prokaryotic origin. In one embodiment the target protein is native to the host cell; in another embodiment the target protein is non-native to the host cell.

One aspect of the invention concerns the use of a prokaryotic vector of the invention, as disclosed here, for enhancing the expression of a recombinant gene encoding a target protein in a prokaryotic host cell.

### IV Methods of Preparing a Microbial Host Cell of the Invention for Expression of a Target Protein

Bacterial transformation may be referred to as a stable genetic change brought about by taking up DNA, and competence refers to the state of being able to take up exogenous DNA. Some bacteria are naturally capable of taking up DNA under laboratory conditions and such species carry sets of genes specifying machinery for bringing DNA across the cell's membrane or membranes, while others have to be induced by laboratory procedures in which cells are passively made permeable to DNA, using conditions that do not normally occur in nature. Chilling cells in the presence of divalent cations such as Ca2+ (in CaCl2) prepares the cell walls to become permeable to plasmid DNA. Cells are incubated on ice with the DNA and then briefly heat-shocked (e.g. 42° C. for 30-120 seconds), which causes the DNA to enter the cell and is a well-known method in the art [Sambrook et al., A Laboratory Manual (1989) CSH]. Electroporation is another way to make cells take up DNA. To persist and be stably maintained in the cell, a plasmid DNA molecule must contain an origin of replication, which allows it to be replicated in the cell independently of the chromosome.

In one aspect, the invention provides a method for producing a recombinant microbial cell having enhanced expression of a recombinant target protein.

In one embodiment, the method for producing a recombinant microbial cell having enhanced expression of a recombinant target protein comprises the steps
a. providing a microbial cell comprising
   i. a gene on the genome of said cell encoding an enzyme having endoribonuclease activity (E.C. 3.1.26), and
   ii. a gene encoding said recombinant target protein,
b. transforming said microbial cell with a prokaryotic vector comprising a recombinant gene encoding a mutant RNase E (E.C. 3.1.26.12) having at least 75% amino acid sequence identity to SEQ ID NO. 2, and wherein the amino acid sequence of said mutant RNase E has one or more amino acid residue substitutions at one or more positions which results in a mutant RNAse E having decreased activity compared to the RNase E of SEQ ID NO. 2,
wherein expression of said target protein is enhanced compared to a cell not transformed with said prokaryotic vector.

In one embodiment, in the method for producing a prokaryotic recombinant microbial cell having enhanced expression of a recombinant target protein, said microbial cell further comprises (iii) a gene encoding a T7 RNA polymerase (E.C. 2.7.7.6), and either said microbial cell or said procaryotic vector comprises a gene encoding a T7 lysozyme (E.C. 3.5.1.28), and expression of the said recombinant gene encoding said target protein is regulated by an inducible T7 promoter

In one preferred embodiment, the method for producing a prokaryotic recombinant microbial cell having enhanced expression of a recombinant target protein comprises the steps
a. providing an E. coli BL21(DE3) strain comprising a gene encoding said recombinant target protein,
b. transforming said microbial cell with pMax

In another embodiment, the method for producing a recombinant microbial cell having enhanced expression of a target protein comprises the steps
a. providing a microbial cell comprising on its genome
   i. a gene on the genome of said cell encoding an enzyme having endoribonuclease activity (E.C. 3.1.26) and
   ii. a first recombinant gene encoding a mutant RNase E (E.C. 3.1.26.12) having at least 75% amino acid sequence identity to SEQ ID NO. 2, and wherein the amino acid sequence of said mutant RNase E has one or more amino acid residue substitutions which results in a mutant RNAse E having decreased activity compared to the RNase E of SEQ ID NO. 2,
b. transforming said microbial cell with a vector comprising a second recombinant gene encoding said target protein,
wherein expression of said target protein is enhanced compared to a cell lacking said first recombinant gene.

### V Method for the Production of a Target Protein

In one aspect, the present invention provides a method for enhancing recombinant protein expression of a target protein, comprising the steps of
a. providing a microbial host cell of the invention as described herein,
b. culturing said host cell in a suitable culture medium, such as a medium that supports growth of said host cell,
c. optionally inducing expression of the recombinant gene encoding the target protein, and
d. optionally isolating and purifying the expressed target protein by well-known techniques.

### VI Method for Screening for Dominant RNase E Mutations

The microbial cell of the Invention comprises wild type RNAse E gene on its genome as well as a mutant RNase E on a plasmid. Hence, the cell produces both wild type and mutant RNase E. For enhancing expression of a target protein, the mutation in the RNase E enzyme presumably facilitates a dominance over the wild type RNase E.

An example of a method for screening for dominant mutations within the RNAse E gene that provide enhanced expression of a recombinant target protein is provided below.

To screen for dominant mutations within the RNAse E gene, a person skilled in the art may use a microbial cell comprising:
i. a gene on the genome of said cell encoding an RNase enzyme (E.C. 3.1.26), and
ii. a gene encoding a recombinant YidC-GFP fusion protein (=target protein use in the screening)

The skilled person should then transform said microbial cell with a prokaryotic vector comprising a recombinant gene encoding a mutant RNase E (that is aimed to be tested/screened) having at least 75% amino acid sequence identity to SEQ ID NO. 2, and wherein the amino acid sequence of said mutant RNase E has an amino acid residue substitution at position X, wherein position x is the residue aimed to be tested/screened. If the tested mutant RNase E exhibits dominance over the genomically encoded wildtype RNase E, fluorescence of the GFP coupled YidC-fusion protein can be observed. If the investigated RNase E mutation is not dominant over the wildtype RNAse E enzyme no fluorescence (or only very little) is observed. The same prokaryotic vector, but lacking the mutant RNase E, may be used as negative control. A dominant RNAse mutant will facilitate enhanced fluorescence compared to the negative control.

In one embodiment, the prokaryotic microbial host cell of the invention comprises
A. a gene encoding an enzyme having endoribonuclease activity (E.C. 3.1.26), wherein said gene is on the genome of said cell,
B. a first recombinant gene encoding a mutant RNase E, and
C. a second recombinant gene encoding a target protein,
   wherein the mutant RNse E enzyme has one or more amino acid residue substitutions which facilitates enhanced expression of the target protein, wherein said amino acid residue substitution is identified and selected by a screening method comprising the steps of
A. expressing the target protein together with a candidate mutant RNAse E comprising a candidate amino acid residue substitution in the host cell,
B. expressing the target protein in a parent cell (from which the host cell was derived) lacking expression of the candidate mutant RNase E,
C. comparing expression levels of the target protein in (a) and (b), and identifying one or more candidate(s) which facilitate enhanced expression of said target protein.

The candidate mutant RNAse may be selected randomly, such as in screening a large library of candidate mutant RNases, or be specific predicted mutations based on knowledge of protein structure, etc.

### VII Measuring Target Protein Expression

The present invention provides a method for enhancing recombinant protein expression as well as a microbial host cell having enhanced expression of a target protein. The enhanced expression is relative to an otherwise identical cell, but which does not comprise a mutant rne* gene, as disclosed herein.

An increase in expression of a target protein may be measured by direct measurement of the amount of the target protein if an assay for such direct measurement of said target protein exists. Alternatively, the expression of the target protein may be measured by fusion of the target protein to a GFP for fluorescence detection. The protein, optionally fused to GFP, may be his-tagged for purification purposes.

### Examples

Bacterial strains: Bacterial strains used in the examples are identified in Table 2. Escherichia col/strain TOP10 (Thermo Fischer Scientific) was used for DNA manipulations, such as plasmid engineering and amplifications. Different strains of E. coli BL21 were used for the expression of genes of interest.

Bacterial strains were grown aerobically at either 37 or 30° C. in Luria-Bertani (LB) broth or agar, supplemented with 50 μg/ml kanamycin, 25 μg/ml chloramphenicol or 100 μg/ml ampicillin depending on the resistance marker of the plasmid used.

TABLE 2
Bacterial strains of the examples

| Bacterial strain | | |
|---|---|---|
| names | Genetic features | Source |
| E. coli | F− mcrA Δ(mrr-hsdRMS-mcrBC) φ80lacZΔM15 | Invitrogen |
| TOP10 | ΔlacX74 nupG recA1 araD139 Δ(ara-leu)7697 | |
| | galE15 galK16 rpsL(Str^{R}) endA1 λ⁻ | |
| E. coli | F⁻ ompT gal dcm lon hsdS_{B}(r_{B}⁻m_{B}⁻) [malB⁺]_{K-12}(λ^{S}) | Invitrogen |
| BL21 | | |
| E. coli | F⁻ ompT gal dcm lon hsdS_{B}(r_{B}⁻m_{B}⁻) λ(DE3 | Invitrogen |
| BL21(DE3) | [lacl lacUV5-T7p07 ind1 sam7 nin5]) [malB⁺]_{K-12}(λ^{S}) | |
| | an E. coli B strain with DE3, a λ prophage | |
| | carrying the T7 RNA polymerase gene and lacl^{q} | |
| | Transformed plasmids containing T7 promoter- | |
| | driven expression are repressed until IPTG | |
| | induction of T7 RNA polymerase from a lac | |
| | promoter. | |
| E. coli | F⁻ ompT gal dcm lon hsdS_{B}(r_{B}⁻m_{B}⁻) λ(DE3 | Invitrogen |
| BL21Star(DE3) | [lacl lacUV5-T7p07 ind1 sam7 nin5]) [malB⁺]_{K-12}(λ^{S}) | |
| | rne131 | |
| | The strain carries a mutated rne gene (rne131) | |
| | which encodes a truncated RNase E enzyme | |
| E. coli | F⁻ ompT gal dcm lon hsdS_{B}(r_{B}⁻m_{B}⁻) [malB⁺]_{K-12}(λ^{S}) | This study |
| Evo21(DE3) | The strain carries a mutated rne gene which | |
| | encodes a truncated RNase E enzyme | |

Plasmids: Plasmids used in the examples are identified in Table 3. The construction of plasmids and transformation into the relevant microbial host cell were performed using standard molecular biology techniques recognized and practised without difficulty by a person skilled in the art, such as the techniques described in Sambrook et al. [A Laboratory Manual (1989) CSH].

TABLE 3
Plasmids and genes of the examples

| Plasmid/Vector | | |
|---|---|---|
| name | Genetic features | Source |
| pLysS | CmR (SEQ ID No.: 30) | Studier, 1991 |
| (SEQ ID No.: 29) | cat promoter (SEQ ID No.: 32) | |
| | P15A ori (SEQ ID No.: 33) | |
| | Φ3.8 promoter (SEQ ID No.: 34) | |
| | 3.5 (T7 lysozyme) (SEQ ID No.: 35) | |
| | tet promoter (SEQ ID No.: 37) | |
| pMax | pLysS backbone | This study |
| (SEQ ID No.: 38) | rne(D346N) (SEQ ID No.: 39) | |
| | rhaBAD promoter (SEQ ID No.: 41) | |
| | BCD (SEQ ID No.: 42) | |
| | rrnc terminator (SEQ ID No.: 43) | |
| | T7 terminator (SEQ ID No.: 44) | |
| pMax-noLysS | CmR (SEQ ID No.: 30) | This study |
| (SEQ ID No.: 45) | cat promoter (SEQ ID No.: 32) | |
| | P15A ori (SEQ ID No.: 33) | |
| | tet promoter (SEQ ID No.: 37) | |
| | rne(D346N) (SEQ ID No.: 39) | |
| | rhaBAD promoter (SEQ ID No.: 41) | |
| | BCD (SEQ ID No.: 42) | |
| | rrnc terminator (SEQ ID No.: 44) | |
| pET28a+ | T7 promoter (SEQ ID No.: 47) | Vendor EMD |
| (SEQ ID No.: 46) | Lac operator (SEQ ID No.: 48) | Biosciences |
| | f1 ori (SEQ ID No.: 49) | addgene.org/vector- |
| | KanR (SEQ ID No.: 50) | database/2565/ |
| | pBR322_ori(SEQ ID No.: 52) | |
| | bom (SEQ ID No.: 53) | |
| | rop (SEQ ID No.: 54) | |
| | lacI promoter (SEQ ID No.: 55) | |
| | lacI (SEQ ID No.: 56) | |
| pP450 | pET28a+ backbone | Vazquez-Albacete et |
| (SEQ ID No.: 58) | Membrane protein P450-GFP-His8 | al, 2016 |
| | (SEQ ID No.: 59) | |
| pHtpX | pET28a+ backbone | Daley et al, 2005 |
| (SEQ ID No.: 61) | Membrane protein HtpX-GFP-His8 | |
| | (SEQ ID No.: 62) | |
| pYqik | pET28a+ backbone | Daley et al, 2005 |
| (SEQ ID No.: 64) | Membrane protein YqiK-GFP-His8 (SEQ | |
| | ID No.: 65) | |
| pYidC | pET28a+ backbone | This study |
| (SEQ ID No.: 67) | Membrane protein YidC-GFP-His8 (SEQ | |
| | ID No.: 68) | |
| | hp6 (SEQ ID No.: 70) | |
| | AmpR (SEQ ID No.: 71) | |
| pMax-truncV489 | pMax backbone | This study |
| (SEQ ID No.: 73) | rne(D346N) truncated at V489 (SEQ | |
| | ID No.: 74) | |
| pMax-truncL529 | pMax backbone | This study |
| (SEQ ID No.: 76) | rne(D346N) truncated at L529 (SEQ ID | |
| | No.: 77) | |
| pMax-D346X | pMax backbone | This study |
| (SEQ ID No.: 79) | rne(D346X) (SEQ ID No.: 80) | |
| pMax-rne^{WT} | pMax backbone | This study |
| (SEQ ID No.: 81) | rne wildtype (SEQ ID No.: 2) | |
| pMax-rne^{STAR} | pMax backbone | This study |
| (SEQ ID No.: 82) | rne131 present in BL21Star(DE3) (SEQ | BL21Star(DE3): Kido |
| | ID No.: 83) | et al, 1996 |
| pMax-rne^{Evo21} | pMax backbone | This study |
| (SEQ ID No.: 85) | rne version present in Evo21(DE3) | |
| | (SEQ ID No.: 86) | |

Plate reader experiments. For growth and protein production assays, strains were cultured overnight in 5 mL LB liquid growth medium. Dilutions of 1:50 were grown aerobically for 24 hours in 96-well plates at 37° C. and 200 rpm using Gas Permeable Adhesive Seal (Thermo Fisher Scientific, Waltham, MA, USA) to avoid evaporation. 1 mM IPTG was added at OD₆₀₀=0.3. Growth (absorbance at 600 nm) and fluorescence (GFP: excitation at 485 nm, emission at 528 nm) was measured in 20 min intervals while continuous shaking using a Synergy H1 plate reader (BloTek Instruments, Winooski, VT, USA). All measurements were performed in triplicate.

Statistical analysis. All experiments were performed in triplicate. Error bars and significance values were calculated using the program PRISM. Error bars indicated represent the average squared deviation from the mean (SD). A one-way ANOVA with Dunnett's multiple comparison test was employed to evaluate differences in expression levels of recombinant protein between Evo21(DE3) and other expression hosts. P values<0.05 were accepted as statistically significant. The different significance levels indicated as stars in figures correspond to p-value<0.05 (*), p<0.01 (**), p<0.001 (***), and p<0.0001 (****).

### Example 1: Rne Point Mutation on Auxiliary Plasmid Increases Protein Production

As discussed previously, truncation of the rne locus, such as rne131 in the commercially available BL21Star (DE3) resulting in the RNase E polypeptide lacking its non-catalytic region (while retaining amino acid residues 1-584), causes a bulk stabilization of mRNA degradation, including mRNA produced by T7RNAP (Lopez et al 1999).

Similarly, the present inventors, while evolving a BL21(DE3) strain by tailored evolution to overcome the challenges of protein production toxicity, have identified a truncated me mutant highly efficient in protein production. This strain was named Evo21(DE3) and comprises a truncation of the encoded 1061-residue E. coli endoribonuclease RNase E after amino acid 702 and therefore a polypeptide lacking the last 359 residues of its C-terminus (see FIG. 1A). Evo21(DE3) was isolated from outgrowing bacterial colonies on week-old agar plates. This combined with the knowledge that dominant rne mutants have previously been observed (Briegel et al 2006) implied that different rne variants could be studied by simple co-expression from a plasmid in the presence of the wildtype me on the genome.

Membrane protein YidC was produced as a C-terminal GFP-His8 fusion protein from a pET28a+ derived expression vector (pYidC: SEQ ID NO.: 67), as shown in Table 3 and described by Drew et al 2006. The expression vector comprises a lacI gene, under the control of its native lacI promoter, encoding the lac repressor that binds to a lacO (operator) upstream of the target YidC gene and blocks its expression. When the target gene is operably linked to a T7 promoter (as in pET28a+), then the expression of the target gene is first induced upon the addition of IPTG.

Plasmid pLysS (SEQ ID NO. 29) comprising a gene encoding T7 lysozyme (SEQ ID NO. 36) was utilised to limit basal T7 RNAP expression (Studier et al 1991). Plasmid pLysS was chosen to function as a backbone for co-expression of different rne variants to avoid the cellular burden of having three plasmids present simultaneously. All rne genes expressed on the pLysS backbone were cloned seamlessly in between a lysS terminator and a T7 ϕ3.8 promoters controlling T7 lysS expression flanked by additional terminator sequences: T7 terminator (upstream) and rrnc terminator (downstream).

To compare different variant of the rne gene at different expression levels, the me variants were cloned in front of the rhamnose-inducible rhaBAD promoter on the pLysS plasmid backbone—see illustration pMAX-rne^{X} in FIG. 2A, where rne” is either a full-length rne (rne^{wt}) and truncated rne (rne^{STAR} and rne-^{Evo21}) were successfully cloned. Further, a spontaneous mutant: rne^{D345N} was included in the study.

rne^{wt} encodes full-length E. coli RNase E (SEQ ID NO.: 2). Truncated rne^{STAR} encodes truncated E. coli RNase E polypeptide amino acids 1-584 (SEQ ID NO.: 84). Truncated rne^{Evo21} encodes truncated RNase E polypeptide amino acids 1-702 (SEQ ID NO.: 87).

Mutant rne^{D346N} encodes full-length RNase E polypeptide comprising amino acid substitution D346N (SEQ ID NO.: 40).

E. coli BL21(DE3) cells co-expressing pLysS and pYidC fall to express detectable levels of the YidC-GFP fusion protein. When the pLysS plasmid was substituted with a pLysS plasmid expressing the full-length rne^{wt} gene or the truncated rne^{STAR} or rne-^{Evo21} genes neither had a positive effect on YidC-GFP expression level. However, the mutant rne^{D346N} was found to significantly enhance YidC-GFP production (see FIG. 2B).

### Example 2: Rne Point Mutation on Auxiliary Plasmid Increases Protein Production Compared to Genomic Rne Truncation

Expression of the rne^{D346N} mutant gene on a plasmid (pMax) in E. coli strain BL21(DE3) comprising the native rne gene of the genome was tested further to establish that its positive effect on heterologous protein production levels was not limited to the expression of YidC as illustrated in example 1, but that the effect of the rne^{D346N} gene in pMax is more versatile.

pMax (SEQ ID NO.: 38) comprises the pLysS as the backbone, with the rne^{D346N} mutant gene operably linked to the rhaBAD promoter on the pLysS plasmid backbone, see illustration in FIG. 3A. The LysS encoding T7 lysozyme helps limit basal T7 RNA polymerase expression.

Membrane proteins P450, HtpX, YqiK, and YidC were produced as C-terminal GFP-His8 fusion proteins from a pET28a+ derived expression vector (pP450: SEQ ID NO.: 58, pHtpX: SEQ ID NO.: 61, pYqiK: SEQ ID NO.: 64, and pYidC: SEQ ID NO.: 67), see illustration in FIG. 3A.

The effect of co-expression of pMax (comprising the rne^{D346N} mutant) compared to the pLysS control, in BL21(DE3) strains producing membrane proteins P450, HtpX, YqiK, and YidC from expression vectors pP450, pHtpX, pYqiK, and pYidC can be seen in FIG. 3B. The data clearly showed that co-expression of the rne (D346N) mutant significantly improved protein production and that the effect was not gene-specific.

This was further compared to the expression of the proteins in strain BL21Star (DE3)pLysS (comprising genomic truncated rne gene and expressing plasmid pLysS). It was found that BL21(DE3)pMax comprising the rne^{D346N} mutant provided on the pLysS plasmid could increase protein production even further than the commercially available solution E. coli host: BL21Star (DE3)pLysS (see FIG. 3B).

This provides a simple tool, in the form of an auxiliary plasmid: pMax, that can be transformed into other strains, to improve protein production titers.

### Example 3: Synergy by Co-Expression of Rne Point Mutation and LysS

pMax harbours both a lysS gene encoding T7 lysozyme and a rne^{D346N} gene encoding an RNase E enzyme having an aspartate to asparagine substitution of amino acid residue 346. The T7 lysozyme—commonly expressed via the pLysS plasmid—counteracts the inherent leakiness of the T7 promotor when controlling expression of a GOI (gene of interest) upon IPTG induction in any E. coli strain harbouring a genomically integrated gene encoding T7 RNAP (T7 RNA polymerase). The presence of the T7 RNAP gene in such strains is annotated as “DE3”, e.g. In BL21(DE3). To investigate whether the combination of the lysS and rne^{D346N} gene is essential to obtain the increase in protein production observed when co-expressing plasmid pMax with a pET/T7 expression vector such as demonstrated in example 2—expression levels of YidC were compared when co-expressing plasmids having either the lysS gene individually (pLysS: SEQ ID NO.: 29) or the rne^{D346N} gene individually (pMax-noLysS: SEQ ID NO.: 45) or harbouring both lysS and rne^{D346N} (pMax: SEQ ID NO.: 38) with a pET/T7 expression vector driving the expression of yidC membrane protein (pYidC: SEQ ID NO.: 67).

As seen in FIG. 4, pMax-noLys (rne^{D346N} alone) provides a statistically significant improvement in protein expression compared to pLysS (lysS alone), while pMax (the combination of lysS and rne^{D346N}) provides an even greater improvement compared to both pMax-noLys and pLysS, and thereby demonstrates a true synergy between rne^{D346N} and LysS.

### Example 4: Rne Truncation and Point Mutation Combined on Auxiliary Plasmid

The effect of expressing an RNase enzyme having a combination of the amino acid mutation D346N along with truncation of the C-terminal domain of the enzyme was investigated. Two genes encoding different truncated rne^{D346N} versions of RNase E harboured on pMax only comprising the N-terminal catalytic half of the enzyme were prepared to explore if such truncated versions of the full rne^{D346N} gene on plasmid pMax would confer the same “protein production-enhancing effect” as observed with the single rne^{D346N} mutant on pMax. The two truncated rne versions encode amino acid residues 1-489 (pMax_truncV489: SEQ ID NO.:75) and 1-529 (pMax_truncL529: SEQ ID NO.:78) of the full rne, respectively, while still harbouring mutation D346N. The truncated rne^{D346N} versions harboured on pMax were co-expressed in BL21(DE3) with the pET/T7 expression vector driving the expression of yidC membrane protein (pYidC: SEQ ID NO.: 67). As seen in FIG. 5, the pMax carrying the truncated rne^{D346N} versions perform better than simply pLysS, but the non-truncated rne^{D346N} outperforms the truncated versions.

A spontaneous mutant of truncL529-rne^{D346N} at position A441V arose during the experiment. This mutant had elevated protein production closer to pMax (full-length rne^{D346N} gene) levels (see FIG. 5).

### Example 5: Rne Genomic Truncation Combined with Point Mutation on Auxiliary Plasmid

BL21Star comprises genomic mutant rne131 which encodes a truncated RNase E lacking the C-terminal region. pMax was co-expressed with pYidC in both E. coli BL21(DE3) and BL21Star (DE3) to examine the effect of having a full length vs a truncated RNase E expressed by the host cell genome in combination with the mutant RNase E encoded by rne*. FIG. 6 clearly shows that the expression of YidC in BL21(DE3) is significantly greater than expression in BL21Star (DE3). It was further shown that the expression of YidC when co-expressed with pMax is in fact no improvement over simply co-expression with pLysS.

### Example 6: Role of Different Amino Acids Substitution: Rne^{D346X}

The importance of the specific nature of the amino acid residue replacing the native aspartate at position 346 in rne encoded RNase E was investigated. For this, a site-directed mutagenesis library was created comprising a range of possible amino acid substitutions in position D346. For this, plasmids similar to pMax were created in which the aspartate encoded within the rne gene in amino acid position 346 was exchanged to amino acids Phe, Leu, Ile, Val, Ser, Pro, Thr, Ala, Tyr, His, Gin, Lys, Glu, Cys and Gly. This pMax (rne^{D346X}) mutant library was co-expressed with pYidC in BL21(DE3) and expression of YidC was compared to co-expression of pLysS with pYidC. As seen in FIG. 7, all tested rne^{D346X} substitutions on pMax outperform pLysS and enhance expression of YidC, from which can be concluded that other amino acid substitutions in the same location D346 in the rne gene lead to the same effect described for the D346N mutation in pMax.

### Example 7: Various Rne Mutations on Auxiliary Plasmids Increase Protein Production

Site-directed mutagenesis (see Table 4) was employed to prepare different substitutions in the RNAse E enzyme predicted herein to alter RNase E functionality (see Table 1). Similar to example 6, a site-directed mutagenesis library was created comprising the amino acid substitutions specified in Table 4. This second pMax (rne^{X}) mutant library was co-expressed with pYidC in BL21(DE3) and expression of YidC was compared to co-expression of pMax (harbouring rne^{D346N}) with pYidC, co-expression of pLysS with pYidC as well as co-expression of pLysS-rneWT with pYidC. As seen in FIG. 8, several rne^{X} substitutions on pMax outperform pLysS and pLysS-rneWT, and enhance expression of the target protein YidC. Hence, amino acid substitutions in several different locations in the rne gene lead to the same effect described for the D346N mutation in pMax.

The data support that mutations in the DNAse I like domain, such as exemplified by E297A, D303N, E325A, R337A, D346N, and R373A in FIG. 8, are favourable in regard to modulating the activity of the mutant RNAse E, leading to enhanced expression of the target protein YidC.

The data also support that mutations which effect the metal iron chelating ability of the RNase E enzyme, such as exemplified by E297A, D303N, E325A, R337A, and D346N in FIG. 8, are favourable in regard to modulating the activity of the mutant RNAse E, leading to enhanced expression of the target protein YidC.

The data further support that mutations which modify an RNA contact point of the RNase E enzyme, such as exemplified by F57A, F67A, K112A in FIG. 8, are favourable in regard to modulating the activity of the mutant RNAse E, leading to enhanced expression of the target protein YidC.

The data further support that mutations in the 5′ sensor pocket, such as the pocket anchors, such as exemplified by V128A and R373A in FIG. 8, are favourable in regard to modulating the activity of the mutant RNAse E, leading to enhanced expression of the target protein YidC.

Finally, the data further support that a mutation at A441 (FIG. 8) is favourable in regard to modulating the activity of the mutant RNAse E, leading to enhanced expression of the target protein YidC.

TABLE 4
RNase E substitutions explored

| Position | Substitution | Codon change |
|---|---|---|
| Glu-297 | Ala | GCG |
| Asp-303 | Asn | GCG |
| Glu-325 | Ala | GCG |
| Val-128 | Ala | GCG |
| Arg-337 | Ala | GCG |
| Phe57 | Ala | GCG |
| Phe67 | Ala | GCG |
| Lys112 | Ala | GCG |
| R373 | Ala | GCG |
| A441 | Val | ACC |

¹Garey et al 2009,
²Callaghan et al 2005,
³Kim et al 2014.

### Example 8: Double Rne Mutation on Auxiliary Plasmid

Site-directed mutagenesis (similar to Example 7) was employed to prepare double substitution in the RNAse E enzyme: rne^{D346N, X}. These double mutans were co-expressed with pYidC in BL21(DE3), and expression of YidC was compared to co-expression of pMax (harbouring rne^{D346N}) with pYidC, and co-expression of pLysS-rneWT with pYidC. As seen in FIG. 9, a slight improved effect was obtained for some of the double mutants.

### Example 9: Improving Translation Initiation Region to Regulate Efficiency of Translation

Since both RNase E activity (altered via mutations) and the expression level of the enzyme itself (altered via expression optimisation) is predicted to be of importance for the underlying mechanism of pMax, a TIR (Translation Initiation Region) library is created using site-directed random mutagenesis. The efficiency of translation initiation is dependent on the nucleotide sequence of the TIR, which comprises the Shine-Dalgarno (SD) sequence and the regions up- and downstream of the SD and is often the rate-limiting step when it comes to protein production in bacteria. Nucleotide changes within this region can affect RNase E production levels greatly as they affect mRNA secondary structure and binding of the ribosome. The TIR ahead of the rne gene on pMax is randomized to obtain pMax versions expressing rne at different levels, which can potentially further enhance protein production titers using pMax. The creation of the TIR library, moreover, enables the isolation of pMax plasmid variants in which rne* expression levels can be precisely tuned via the already existing rhamnose-inducible promotor (PrhaBAD). For the creation of the TIR library, the six nucleotides upstream of the ATG codon of the the BCD located downstream of the PrhaBAD promotor controlling expression of the rne* gene on plasmid pMax are randomised (NNNNNN). Additionally, the 2^{nd} and 3^{rd} codon of the BCD will be replaced by AARGCN. The pMax mutant TIR library is then co-transformed into BL21(DE3) along with the pYidC expression vector, and colonies are plated on LB agarose plates containing different concentrations of the inducer L-rhamnose (0.1-5 mM). Cells producing high amounts of the YidC-GFP fusion protein will be identified via green fluorescence visible under UV light and will be isolated and sequenced. This way, individual TIR sequences with optimal rne expression levels are identified based on their stimulatory effect on YidC protein production. Additionally, colonies that show YidC expression on 1 mM rha concentration plates will be restreaked on a agarose plate dilution series ranging from 0 to 5 mM rhamnose to identify TIRs that allow tight tunability of rne* expression on pMax. Such candidates will express high YidC levels on agarose plates containing 5 mM rhamnose and will not show YidC expression when plated on 0 mM rha, respectively.

### Example 10: Effect of Plasmid Copy Number

The pLysS plasmid backbone currently used as the basis for pMax is replaced by other plasmid backbones propagated by different origins of replication (ori). To this end, rne* and lysS gene along with their respective regulatory elements (respective promoter and terminator elements) are transferred onto alternative vector backbones maintained in the cell with a) high copy number (e.g. plasmids carrying oris pColE1/F1 (300-500 copies) or pUC derivative pMB1 (500-700 copies)) or b) low copy number (e.g. plasmids harbouring oris pSC101 (5 copies), RK2 (4-7), p15A (10 copies), R6k (15-20 copies), ColE1 (15-20 copies), pMB1 (15-20 copies) or pBR322 (15-20 copies)). The plasmid backbone pMax variants are co-transformed along with pYidC into BL21(DE3), and optimal backbones are identified based on their stimulatory effect on YidC protein production. It is expected, that the low copy plasmid backbone pMax variants will outperform high copy vector versions and pLysS similarly to pMax enhancing expression of the target protein YidC, from which will be concluded that the plasmid backbone copy number is a limiting factor for an optimal tuning of RNase E* levels in the production host cell.

### Example 11: Protein Production in Other DE3-Strains

It was investigated whether pMax has a positive effect on protein production in another DE3 strain (other than BL21(DE3))—hence another strain that performs T7 RNAP-dependent gene expression. Effect of pMax co-expression on Proteinase K production (activity expressed in cytosol) in E. coli Shuffle (DE3) was compared to co-expression using pLysS or pET-empty vector (FIG. 10), finding a positive effect for pMax.

### Example 12: Protein Production in Non DE3 Strains

It is investigated whether pMax or the co-expression of solely rne (D346N) (pMax-nolysS) has a positive effect on protein production in non DE3 strains—hence strains that do not perform T7 RNAP-dependent gene expression. Expression levels and growth of E. coli BL21(DE3) and E. coli non-DE3 strains: BL21 and K12 MG1655 are compared, when co-expressing pMax and a GOI such as yidC under the control of non-T7-dependent promoter systems such as the rhamnose-inducible PrhaBAD, the arabinose-Inducible promoter ParaBAD, the tryptophan-inducible promotor Ptrc, the tetracycline-Inducible promoter Ptet, the IPTG-inducible promotor (Ptac) and the heat-Inducible promoter PL.

### Example 13: Expression of Challenging/Toxic Proteins (YldC) Vs Non-Toxic Proteins (GFP)

The heterologous production of GFP-fusion protein YidC-GFP and GFP in BL21(DE3) was investigated when co-expressing either auxiliary plasmid pLysS (lysS) or pMax (lysS, meD346N). As seen in FIG. 11, pMax facilitates an improvement in expression compared to pLysS.

### REFERENCES

Angius, F.; Illoaia, O.; Amrani, A.; Suisse, A.; Rosset, L.; Legrand, A.; Abou-Hamdan, A.; Uzan, M.; Zito, F.; Miroux, B. A Novel Regulation Mechanism of the T7 RNA Polymerase Based Expression System Improves Overproduction and Folding of Membrane Proteins. Sci. Rep. 2018, 8 (1), 1-11. doi.org/10.1038/s41598-018-26668-y.
Baumgarten, T.; Schlegel, S.; Wagner, S.; Löw, M.; Eriksson, J.; Bonde, I.; Herrgård, M. J.; Heipieper, H. J.; Nørholm, M. H. H.; Slotboom, D. J.; et al. Isolation and Characterization of the E. Coli Membrane Protein Production Strain Mutant56(DE3). Sci. Rep. 2017, 7 (March), 1-14. doi.org/10.1038/srep45089.
Bonde, M. T., Pedersen, M., Klausen, M. S., Jensen, S. I., Wulff, T., Harrison, S., et al. (2016). Predictable tuning of protein expression in bacteria. Nat. Methods 13. doi:10.1038/nmeth.3727.
Briegel, K. J.; Baker, A.; Jain, C. Identification and Analysis of Escherichia Coli Ribonuclease E Dominant-Negative Mutants. Genetics 2006, 172 (1), 7-15. doi.org/10.1534/genetics.105.048553.
Callaghan, A. J.; Marcaida, M. J.; Stead, J. A.; McDowall, K. J.; Scott, W. G.; Luisi, B. F. Structure of Escherichia Coll RNase E Catalytic Domain and Implications for RNA Turnover. Nature 2005, 437 (7062), 1187-1191. doi.org/10.1038/nature04084.
Daley et al: Global topology analysis of the Escherichia coli inner membrane proteome. Science Vol 308, Issue 5726, 27 May 2005 DOI: 10.1126/science.1109730
Drew, D.; Lerch, M.; Kunji, E.; Slotboom, D. J.; de Gier, J. W. Optimization of Membrane Protein Overexpression and Purification Using GFP Fusions. Nat. Methods 2006, 3 (4), 303-313. doi.org/10.1038/nmeth0406-303.
Fellmeier et al.: Green fluorescent protein functions as a reporter from protein localization in Escherichia coli. American Society for Microbiology. Journal of Bacteriology. 2000 DOI: 10.1128/JB.182.14.4068-4076.2000
Garrey, S. M.; Blech, M.; Riffell, J. L.; Hankins, J. S.; Stickney, L. M.; Diver, M.; Hsu, Y. H. R.; Kunanithy, V.; Mackie, G. A. Substrate Binding and Active Site Residues in RNases E and G: Role of the 5′-Sensor. J. Blol. Chem. 2009, 284 (46), 31843-31850. doi.org/10.1074/jbc.M109.063263.
Kido, M.; Yamanaka, K.; Mitani, T.; Niki, H.; Ogura, T.; Hiraga, S. RNase E Polypeptides Lacking a Carboxyl-Terminal Half Suppress a MukB Mutation in Escherichia Coli. J. Bacteriol. 1996, 178 (13), 3917-3925. doi.org/10.1128/jb.178.13.3917-3925.1996.
Kim, D. et al. Modulation of RNase E activity by alternative RNA binding sites. PLoS One 9, (2014).
Kwon, S. K.; Kim, S. K.; Lee, D. H.; Kim, J. F. Comparative Genomics and Experimental Evolution of Escherichia Coll BL21(DE3) Strains Reveal the Landscape of Toxicity Escape from Membrane Protein Overproduction. Sci. Rep. 2015, 5 (October), 16076. doi.org/10.1038/srep16076.
Lopez, P. J.; Marchand, I.; Joyce, S. A.; Dreyfus, M. The C-Terminal Half of RNase E, Which Organizes the Escherichia Coli Degradosome, Participates in MRNA Degradation but Not RRNA Processing in Vivo. Mol. Microbiol. 1999, 33 (1), 188-199. ol.org/10.1046/j.1365-2958.1999.01465.x.
Mardle et al: A structural and biochemical comparison of Ribonuclease E homologues from pathogenic bacteria highlights species-specific properties. Scientific Reports volume 9, Article number: 7952 (2019). doi.org/10.1038/s41598-019-44385-y
Miroux & Walker: Over-production of proteins in Escherichia coli mutant hosts that allow synthesis of some membrane proteins and globular proteins at high levels, 289-298 (1996).
Shahbabian et al: RNase Y, a novel endoribonuclease, initiates riboswitch turnover in Bacillus subtilis. EMBO J (2009)28:3523-3533 doi.org/10.1038/emboj.2009.283
Studier, F. W. Use of Bacteriophage T7 Lysozyme to Improve an Inducible T7 Expression System. J. Mol. Biol. 1991, 219 (1), 37-44. doi.org/10.1016/0022-2836(91)90855-Z.
Vazquez-Albacete et al.: An expression tag toolbox for microbial production of membrane bound plant cytochromes P450. Biotechnology and Bioengineering Vol 144, Iss 4, pp 751-760. April 2017. doi: 10.1002/bit.26203

### ITEMS OF THE INVENTION

1. A prokaryotic microbial host cell for recombinant expression of a target protein, said cell comprising
a. a gene encoding an enzyme having endoribonuclease activity (E.C. 3.1.26), said enzyme preferably having at least 75% amino acid sequence identity to SEQ ID NO. 2, wherein said gene is on the genome of said cell,
b. a first recombinant gene encoding a mutant RNase E, wherein the amino acid sequence of said mutant RNase E has at least 75% sequence identity with SEQ ID NO. 2, and wherein the amino acid sequence of said mutant RNase E has one or more amino acid residue substitutions at one or more positions selected from D346, E297, D303, N305, E325, R337, D349, V128, R169, T170, F57, F67, K112, G124, R141, R142, R373, and A441 relative to SEQ ID NO. 2, and
c. a second recombinant gene encoding said target protein,
wherein expression of said target protein is enhanced compared to a cell lacking said first recombinant gene.

2. A host cell according to item 1, wherein said mutant RNase E has one or more amino acid residue substitutions at one or more positions selected from D346, E297, D303, N305, E325, R337, and D349.

3. A host cell according to items 1 or 2, wherein said amino acid residue substitution at position 346 of said mutant RNase E relative to SEQ ID NO. 2 is asparagine.

4. A host cell according to any of items 1-3, wherein said cell further comprises a first prokaryotic vector, and wherein said first recombinant gene encoding said mutant RNase E is comprised on said first prokaryotic vector.

5. A host cell according to any of items 1-4, wherein said cell further comprises
d. a gene encoding a T7 RNA polymerase (E.C. 2.7.7.6),
e. optionally a gene encoding a T7 lysozyme (E.C. 3.5.1.28) and wherein expression of said second recombinant gene is regulated by an Inducible T7 promoter.

6. A host cell according to item 5, wherein said gene encoding said T7 lysozyme is located on the first prokaryotic vector, and wherein said second recombinant gene encoding said target gene is located on a second prokaryotic vector.

7. A host cell according to any one of items 1-4, wherein expression of said second recombinant gene is regulated by an Inducible promoter selected from rhaBAD promoter, araBAD promoter, Ptrc promotor, Ptet promoter, Ptac promoter, and PL promoter.

8. A host cell according to any one of items 1-7, wherein said target protein is a protein the expression of which is enhanced by at least 10% compared to expression of said protein in the same host cell lacking said first recombinant gene.

9. A host cell according to any one of items 1-8, wherein said cell is selected from E. coli, Bacillus subtilis, Bacillus licheniformis, and Pseudomonas putida.

10. A prokaryotic vector comprising
a. a gene encoding a mutant RNase E (E.C. 3.1.26.12) having at least 75% amino acid sequence identity to SEQ ID NO. 2, wherein the amino acid sequence of said mutant RNase E has one or more amino acid residue substitutions at one or more positions selected from D346, E297, D303, N305, E325, R337, D349, V128, R169, T170, F57, F67, K112, G124, R141, R142, R373, and A441 relative to SEQ ID NO. 2, and
b. a gene encoding a T7 lysozyme (E.C. 3.5.1.28)

11. A prokaryotic vector according to item 10, wherein amino acid residue at position 346 of said mutant RNase E relative to SEQ ID NO. 2 is asparagine.

12. Use of the prokaryotic vector according to item 10 or 11 for enhancing expression of a recombinant gene encoding a target protein in a host prokaryotic microbial cell.

13. A method for the production of a target protein comprising culturing in a suitable culture medium, a host cell according to any one of items 1-9, optionally Inducing expression of said target protein, followed by Isolation and purification of the expressed target protein.

14. A method for producing a prokaryotic recombinant microbial cell having enhanced expression of a recombinant target protein,
a. providing a prokaryotic microbial cell comprising (I) a gene encoding an enzyme having endoribonuclease activity (E.C. 3.1.26), said enzyme preferably having at least 75% amino acid sequence identity to SEQ ID NO. 2, wherein said RNase gene is on the genome of said cell, and (ii) a gene encoding said recombinant target protein,
b. transforming said microbial cell with a prokaryotic vector comprising a recombinant gene encoding a mutant RNase E (E.C. 3.1.26.12) having at least 75% amino acid sequence identity to SEQ ID NO. 2, and wherein the amino acid sequence of said mutant RNase E has one or more amino acid residue substitutions at one or more positions selected from D346, E297, D303, N305, E325, R337, D349, V128, R169, T170, F57, F67, K112, G124, R141, R142, R373, and A441 relative to SEQ ID NO. 2,
wherein expression of said target protein is enhanced compared to a cell not transformed with said prokaryotic vector.

15. Method for producing a prokaryotic recombinant microbial cell having enhanced expression of a recombinant target protein according to item 14, wherein said microbial cell further comprises (III) a gene encoding a T7 RNA polymerase (E.C. 2.7.7.6), wherein expression of the said recombinant gene encoding said target protein is regulated by a T7 promoter, and wherein said microbial cell or said procaryotic vector optionally comprises a gene encoding a T7 lysozyme (E.C. 3.5.1.28).

1. A prokaryotic microbial host cell for recombinant expression of a target protein, said cell comprising
A. a gene encoding an enzyme having endoribonuclease activity (E.C. 3.1.26), wherein said gene is on the genome of said cell,
B. a first recombinant gene encoding a mutant RNase E, wherein the amino acid sequence of said mutant RNase E has at least 75% sequence identity with SEQ ID NO. 2, and wherein the amino acid sequence of said mutant RNase E has one or more amino acid residue substitution(s) which results in the mutant RNAse E having decreased activity compared to the RNase E of SEQ ID NO. 2, and
C. a second recombinant gene encoding said target protein,
wherein expression of said target protein is enhanced compared to a cell lacking said first recombinant gene.

2. The prokaryotic microbial host cell according to claim 1, wherein the target protein is a toxic protein such as wherein the target protein is a membrane protein.

3. (canceled)

4. The prokaryotic microbial host cell according claim 1, wherein the enzyme having ribonuclease activity (E.C. 3.1.26), encoded by the gene on the genome, is native to the host cell.

5. The prokaryotic microbial host cell according to claim 1, wherein the enzyme having ribonuclease activity (E.C. 3.1.26), encoded by the gene on the genome, is an RNase E enzyme having at least 75% sequence identity with SEQ ID NO. 2.

6. The prokaryotic microbial host cell according to claim 1, wherein the one or more amino acid residue substitutions is at one or more positions selected from D346, E297, D303, N305, E325, R337, D349, V128, R169, T170, F57, F67, K112, G124, R141, R142, or R373 relative to SEQ ID NO. 2.

7. The prokaryotic microbial host cell according to claim 1, wherein the one or more amino acid residue substitutions is at one or more positions in the DNAse I-like domain, such as one or more positions selected from E297, D303, N305, E325, R337, D346, D349, and R373.

8. (canceled)

9. The prokaryotic microbial host cell according to claim 1, wherein the one or more amino acid residue substitutions results in a mutant RNAse E having reduced metal ion chelation ability compared to the RNase E of SEQ ID NO. 2, such as one or more positions selected from D346, E297, D303, E325, R337, and D349 relative to SEQ ID NO. 2.

10. (canceled)

11. The prokaryotic microbial host cell according to claim 1, wherein the one or more amino acid residue substitutions results in a mutant RNAse E having a modified RNA contact point compared to the RNase E of SEQ ID NO. 2, such as one or more positions selected from F57, F67, and K112.

12. (canceled)

13. The prokaryotic microbial host cell according to claim 1, wherein the one or more amino acid residue substitutions is in the 5′ sensor pocket, preferably the pocket ‘anchors’, such as one or more amino acid residue substitutions is at positions V128 and/or R373.

14. (canceled)

15. The prokaryotic microbial host cell according to claim 1, wherein the amino acid residue substitution is A441.

16. The prokaryotic microbial host cell according to claim 1, wherein the amino acid residue substitution facilitates the enhanced expression of said target protein, and wherein said amino acid residue substitution is identified and selected by a screening method comprising the steps of
A. expressing the target protein together with a candidate mutant RNAse E comprising a candidate amino acid residue substitution in the host cell,
B. expressing the target protein in a parent cell (from which the host cell was derived) lacking expression of the candidate mutant RNase E,
C. comparing expression levels of the target protein in (a) and (b), and identifying one or more candidate(s) which facilitate enhanced expression of said target protein.

17. The prokaryotic microbial host cell according to claim 1, wherein said cell further comprises a first prokaryotic vector, and wherein said first recombinant gene encoding said mutant RNase E is comprised on said first prokaryotic vector.

18. The prokaryotic microbial host cell according to claim 1, wherein said cell further comprises
D. a gene encoding a T7 RNA polymerase (E.C. 2.7.7.6),
E. optionally a gene encoding a T7 lysozyme (E.C. 3.5.1.28)
and wherein expression of said second recombinant gene is regulated by an inducible T7 promoter.

19. The prokaryotic microbial host cell according to claim 18, wherein said gene encoding said T7 lysozyme is located on the first prokaryotic vector, and wherein said second recombinant gene encoding said target gene is located on a second prokaryotic vector.

20. The prokaryotic microbial host cell according to claim 1, wherein expression of said second recombinant gene is regulated by an inducible promoter selected from rhaBAD promoter, araBAD promoter, Ptrc promotor, Ptet promoter, Ptac promoter, and PL promoter.

21. The prokaryotic microbial host cell according to claim 1, wherein said target protein is a protein the expression of which is enhanced by at least 10% compared to expression of said protein in the same host cell lacking said first recombinant gene.

22. The prokaryotic microbial host cell according to claim 1, wherein said cell is selected from E. coli, Bacillus subtilis, Bacillus licheniformis, and Pseudomonas putida.

23. A prokaryotic vector comprising
A. a gene encoding a mutant RNase E (E.C. 3.1.26.12) having at least 75% amino acid sequence identity to SEQ ID NO. 2, wherein the amino acid sequence of said mutant RNase E has one or more amino acid residue substitution(s) which results in the mutant RNAse E having decreased activity compared to the RNase E of SEQ ID NO. 2, and
B. a gene encoding a T7 lysozyme (E.C. 3.5.1.28)

24. The prokaryotic vector according to claim 23, wherein the one or more amino acid residue substitutions (i) is at one or more positions in the DNAse I-like domain, (ii) results in a mutant RNAse E having reduced metal ion chelation ability compared to the RNase E of SEQ ID NO. 2. (iii) results in a mutant RNAse E having a modified RNA contact point compared to the RNase E of SEQ ID NO. 2, or (iv) is in the 5′ sensor pocket, such as the pocket ‘anchors’.

25. (canceled)

26. (canceled)

27. (canceled)

28. (canceled)

29. A method for the production of a target protein, comprising culturing in a suitable culture medium, a prokaryotic microbial host cell according to claim 1, expressing said target protein, and optionally isolating the expressed target protein.
